(19) United States
(12) Patent Application Publication
(10) Pub. No.: US 2025/0206826 A1
(43) Pub. Date: Jun. 26, 2025

(54) METHODS OF DEPLETING DISEASE CAUSING AGENTS VIA ANTIBODY TARGETED PHAGOCYTOSIS

(71) Applicant: Dren Bio, Inc., Foster City, CA (US)

(72) Inventors: Panagiotis FOTAKIS, Foster City, CA (US); Chanty Mariategue CHAN, San Francisco, CA (US); Ruo Shi SHI, San Mateo, CA (US); Adam Lewis SALLES, San Bruno, CA (US); Nenad TOMASEVIC, Hillsborough, CA (US)

(73) Assignee: Dren Bio, Inc., Foster City, CA (US)

(21) Appl. No.: 18/975,761

(22) Filed: Dec. 10, 2024

Related U.S. Application Data
(63) Continuation of application No. 17/601,359, filed on Oct. 4, 2021, now abandoned.
(60) Provisional application No. 62/830,139, filed on Apr. 5, 2019.

(51) Int. Cl. C07K 16/28; C07K 16/46

(52) U.S. Cl. CPC C07K 16/2851; C07K 16/468; C07K 2317/31; C07K 2317/73

(57) ABSTRACT

The present disclosure relates to a method of depleting or reducing the numbers of disease-causing agents including host cells, or host cells products, microbes or their products in a human subject upon administration of molecule that causes targeted phagocytosis and comprises a binding domain that binds a specific phagocytotic receptor, such as Dectin-1, and a binding domain that binds a specific disease-causing agent. In a specific embodiment, a method of the disclosure depletes or reduces the number of disease-causing agents in tissues, blood, or bone marrow by targeted phagocytosis.

### CROSS REFERENCE TO RELATED APPLICATIONS

This application is a continuation of U.S. patent application Ser. No. 17/601,359, filed Oct. 4, 2021, which is a national stage application under 35 U.S.C. § 371 of International Application No. PCT/US2020/026721, filed on Apr. 3, 2020, which claims priority to U.S. Provisional Application No. 62/830,139, filed Apr. 5, 2019, the disclosures of each of which are hereby incorporated by reference in their entirety.

### FIELD

The present disclosure relates to methods of depleting or reducing disease-causing agents in humans by targeted phagocytosis.

### BACKGROUND

Professional phagocytes are a subset of white blood cells that commonly refers to monocytes, macrophages, dendritic cells, neutrophils, eosinophils and osteoclasts that specifically recognize and engulf host or foreign agents that are aberrant or cause diseases (Rabinovitch, 1995, Trends in Cell Biol; Arandejelovic, et al, 2015, Nat Immunol; Rosales, et al, 2017 BioMed Research International). Phagocytosis is a major mechanism used to remove pathogens and cell debris. Phagocytosis, defined as the cellular uptake of particulates (>0.5 mm) within a plasma-membrane envelope, is closely related to and partly overlaps the endocytosis of soluble ligands by fluid-phase macropinocytic and receptor pathways (Rosales, et al, 2017 BioMed Research International; Gordon, 2016, Immunity; Tse, et al, 2003, J Biol Chem). The engulfed material is then digested in the phagosome. Bacteria, dead tissue cells, and small mineral particles are all examples of objects that may be phagocytized. Several terms have been applied to mechanisms associated with the uptake of apoptotic cells, also known as efferocytosis, and that of necrotic cells arising from infection and inflammation (necroptosis and pyroptosis) (Henson and Bratton, 2009). The engulfed material is destroyed in the process of phagocytosis through the endo-lysosomal pathway. Dendritic cells and macrophages ingest pathogens by phagocytosis and break them down for antigen presentation to the cells of the adaptive immune system.

Receptors on the plasma membrane of phagocytes that mediate phagocytosis could be divided into non-opsonic and opsonic types. Non-opsonic receptors include lectin-type receptors, Dectin receptors, or scavenger receptors (Freeman and Grinstein, Immunological Reviews, 2014). Some phagocytic pathways require a second signal from pattern recognition receptors (PRRs) activated by attachment to pathogen-associated molecular patterns (PAMPS), which leads to NF-κB activation (Patin, et al, 2018, Semin Cell Dev Biol; Brandt, et al, 2013, PLoS One). Non-opsonic receptors variably expressed by professional phagocytes include lectin-like recognition molecules, such as CD169, CD33, and related receptors for sialylated residues. In addition, phagocytes also express Dectin-1 (a receptor for fungal beta-glucan with well-defined signaling capacity), related C-type lectins (e.g., MICL, Dectin-2, Mincle, and DNGR-1), and a group of scavenger receptors (Asano, et al, 2018, J Biochem, Lock, et al, 2004, Immunobiol). SR-A, MARCO, and CD36 vary in domain structure and have distinct though overlapping recognition of apoptotic and microbial ligands (Freeman and Grinstein, Immunological Reviews, 2014). These promiscuous receptors bind polyanionic ligands and have poorly defined intracellular signaling capacity, perhaps indicating that multi-ligand and receptor interactions are a requirement for uptake. Notably, toll-like receptors (TLRs) are sensors and not phagocytic entry receptors, although they often collaborate with other non-opsonic receptors to promote uptake and signaling (Gordon 2016).

Plasma-membrane receptors can be classified as opsonic, FcRs (activating or inhibitory) for mainly the conserved domain of IgG antibodies, and complement receptors, such as CR3 for iC3b deposited by classical (IgM or IgG) or alternative lectin pathways of complement activation. CR3 can also mediate recognition in the absence of opsonins, perhaps by depositing macrophage-derived complement. Plasma- or cell-derived opsonins include fibronectin, mannose-binding lectin, milk fat globulin (MFG-E8). A list of most common phagocytic receptors is shown in Table A (Rosales 2017).

To date four β-glucan receptors have been identified as candidates mediating anti-fungal phagocytotic activities, namely complement receptor 3 (CR3; CD11b/CD18), lactosylceramide, selected scavenger receptors, and dectin-1 (PGR). Dectin-1 consists of a single C-type, lectin-like, carbohydrate recognition domain, a short stalk, and a cytoplasmic tail possessing an immunoreceptor tyrosine-based activation motif (ITAM). The receptor recognizes particles such as zymosan, Saccharomyces cerevisiae, and heat-killed Candida albicans in a β-glucan-dependent manner (Taylor 2002). Dectin-1 has been clearly shown to be sufficient for activating phagocytosis. It is expressed on myeloid dendritic cells, monocytes, macrophages and B cells.

It would be beneficial to develop targeted removal and degradation of accumulated disease-causing agents without boosting overall phagocytosis. This disclosure provides a solution for the problems and describes other advantages.

All references cited herein, including patent applications, patent publications, and scientific literature, are herein incorporated by reference in their entirety, as if each individual reference were specifically and individually indicated to be incorporated by reference.

### BRIEF SUMMARY

The present disclosure relates to a method of removal and degradation the numbers of disease-causing agents including host cells, or host cells products, microbes or their products in a human subject upon administration of a molecule that comprises a first binding domain that specifically binds to the agent, a second binding domain that binds to a phagocytotic receptor, Dectin-1, expressed on a macrophage and induces phagocytosis, and an immunoglobulin Fc domain. In a specific embodiment, a method of the disclosure depletes or reduces the number of disease-causing agents in tissues, blood, and/or bone marrow by targeted phagocytosis.

In some embodiments, provided herein is a method of reducing number of a disease-causing agent by targeted phagocytosis in a subject, comprising administering to said subject a binding protein comprising a first binding domain that specifically binds to the agent, and a second binding domain that binds to a phagocytotic receptor expressed on a macrophage, monocyte, and/or granulocyte and induces phagocytosis activity of the macrophage, monocyte, and/or granulocyte. In some embodiments, the phagocytotic receptor is Dectin-1, e.g., human Dectin-1. In some embodiments, provided herein is a method of reducing number of a disease-causing agent in a subject, comprising administering to said subject a binding protein comprising a first binding domain that specifically binds to the agent and a second binding domain that binds to Dectin-1. In some embodiments, the binding protein further comprises an immunoglobulin Fc domain. In some embodiments, the binding protein is an antibody (e.g., a multispecific or bispecific antibody). In some embodiments, the subject is a human. In some embodiments, the binding protein is a bispecific antibody comprising a first binding domain that binds to Dectin-1 and a second binding domain that binds to a target antigen expressed by the disease-causing agent. In some embodiments, the subject is a human. In some embodiments, the binding protein is a bispecific antibody comprising a first binding domain that binds to Dectin-1 and a second binding domain that binds to a target antigen expressed by the disease-causing agent, wherein the bispecific antibody has a format shown and/or described in reference to FIG. 1A.

In some embodiments, administration of the binding protein reduces the number of the agent. In some embodiments, administration of the binding protein reduces the number of the agent to below the limit of detection. In some embodiments, administration of the binding protein reduces the number of the agent for at least about 1 week after dosing of the binding protein. In some embodiments, administration of the binding protein reduces the number of the agent within 12 hours, within 24 hours, within 36 hours, or within 48 hours after administration. In some embodiments, reduction of the disease-causing agent is reversible, e.g., after administration of the binding protein is ceased. In some embodiments, administration of the binding protein reduces severity and/or incidence of one or more symptoms in the subject.

In some embodiments, the method results in removal and/or reduction in levels of one or more disease-associated proteins or protein aggregates. In some embodiments, the method results in inhibition of aberrant protein accumulation. In some embodiments, the method results in alleviating or preventing progression of one or more symptoms of a disease, e.g., a neurodegenerative disease, fibrosis, or amyloidosis. In some embodiments, the binding protein is a bispecific antibody comprising a first binding domain that binds to Dectin-1 and a second binding domain that binds to a target protein or protein aggregate.

In some embodiments, the method results in removal and/or reduction in number of cancer, tumor or lymphoma cells. In some embodiments, the method results in alleviating one or more symptoms of cancer and/or preventing progression of cancer. In some embodiments, the binding protein is a bispecific antibody comprising a first binding domain that binds to Dectin-1 and a second binding domain that binds to a target antigen expressed by a cancer cell (e.g., a tumor antigen expressed on the surface of a cancer cell).

In some embodiments, the method results in removal and/or reduction in levels of one or more microbes (e.g., a bacterial cell, fungal cell, protozoan cell, or virus). In some embodiments, the method results in alleviating or preventing progression of one or more symptoms of a disease or infection caused by a microbe (e.g., a bacterial cell, fungal cell, protozoan cell, or virus). In some embodiments, the binding protein is a bispecific antibody comprising a first binding domain that binds to Dectin-1 and a second binding domain that binds to a target antigen expressed by a bacterial cell (e.g., an antigen expressed on the surface of a bacterial cell). In some embodiments, the binding protein is a bispecific antibody comprising a first binding domain that binds to Dectin-1 and a second binding domain that binds to a target antigen expressed by a fungal cell (e.g., an antigen expressed on the surface of a fungal cell). In some embodiments, the binding protein is a bispecific antibody comprising a first binding domain that binds to Dectin-1 and a second binding domain that binds to a target antigen expressed by a protozoan cell (e.g., an antigen expressed on the surface of a protozoan cell). In some embodiments, the binding protein is a bispecific antibody comprising a first binding domain that binds to Dectin-1 and a second binding domain that binds to a target antigen expressed by a virus (e.g., an antigen expressed on the surface of virus).

In some embodiments, the method results in removal and/or reduction in levels of senescent cells and/or their product(s). In some embodiments, the method results in alleviating or preventing progression of ageing, e.g., in one or more age-related symptoms or conditions. In some embodiments, the binding protein is a bispecific antibody comprising a first binding domain that binds to Dectin-1 and a second binding domain that binds to a target antigen expressed by a senescent cell (e.g., an antigen expressed on the surface of a senescent cell).

In some embodiments, the method results in removal and/or reduction in levels of LDL and other agents that induce cardiovascular disease, e.g., arteriosclerosis or familial hypercholesterolemia. In some embodiments, the method results in alleviating or preventing progression of one or more symptoms of a cardiovascular disease, e.g., arteriosclerosis or familial hypercholesterolemia. In some embodiments, the binding protein is a bispecific antibody comprising a first binding domain that binds to Dectin-1 and a second binding domain that binds to a lipoprotein particle (e.g., LDL).

In some embodiments, the method results in removal and/or reduction in levels of mast cells. In some embodiments, the method results in alleviating or preventing progression of one or more symptoms of a mast cell-related disease, e.g., allergy, fibrosis, COPD, asthma, or other immunoproliferative, mast cell-related diseases. In some embodiments, the binding protein is a bispecific antibody comprising a first binding domain that binds to Dectin-1 and a second binding domain that binds to a target antigen expressed by a mast cell (e.g., an antigen expressed on the surface of a mast cell).

In some embodiments, the method results in removal and/or reduction in levels of eosinophils. In some embodiments, the method results in alleviating or preventing progression of one or more symptoms of an eosinophil-related disease, e.g., allergy, fibrosis, COPD, asthma, or other immunoproliferative, eosinophil-related diseases. In some embodiments, the binding protein is a bispecific antibody comprising a first binding domain that binds to Dectin-1 and a second binding domain that binds to a target antigen expressed by eosinophil (e.g., an antigen expressed on the surface of an eosinophil).

In some embodiments, the method results in removal and/or reduction in levels of ILC2 cells. In some embodiments, the method results in alleviating or preventing progression of one or more symptoms of an ILC2-related disease, e.g., allergy, fibrosis, COPD, asthma, or other immunoproliferative, ILC2-related diseases. In some embodiments, the binding protein is a bispecific antibody comprising a first binding domain that binds to Dectin-1 and a second binding domain that binds to a target antigen expressed by an ILC2 cell (e.g., an antigen expressed on the surface of an ILC2 cell).

In some embodiments, the method results in removal and/or reduction in levels of inflammatory immune cells, e.g., in one or more tissues selected from the group consisting of muscles, GI tract, lungs, heart, joints, and brain. In some embodiments, the method results in alleviating or preventing progression of one or more symptoms of myositis, IBD, RA, allergy, fibrosis, COPD, asthma, or other immunoproliferative, inflammatory immune cell-related diseases. In some embodiments, the binding protein is a bispecific antibody comprising a first binding domain that binds to Dectin-1 and a second binding domain that binds to a target antigen expressed by an inflammatory immune cell (e.g., an antigen expressed on the surface of an inflammatory immune cell).

In some embodiments, the binding protein is an antibody; two antibodies or IgGs that are covalently linked; IgG-scFv; intrabody; peptibody; nanobody; single domain antibody; SMTP; multispecific antibody (e.g., bispecific antibodies, diabodies, triabodies, tetrabodies, tandem di-scFV, tandem tri-scFv, ADAPTIR); Fab, Fab′, F(ab′)2, or Fv fragment; Fab′-SH or F(ab′)2 diabody; linear antibody; scFv antibodies; VH antibody; or multispecific antibody formed from antibody fragments. In some embodiments, one or more binding domain(s) of the binding protein are non-human, chimeric, humanized, or human. In some embodiments, one or more binding domain(s) of the binding protein are humanized or human. In some embodiments, both binding domains of the binding protein are non-human, chimeric, humanized, or human. In some embodiments, both binding domains of the binding protein are humanized or human.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1A provides schematic diagrams of antibody molecules for targeted phagocytosis, in accordance with some embodiments. A bispecific antibody that binds to Dectin-1 (d) and a disease-causing agent (a) is shown in FIG. 1A at panel A. Examples of IgG-scFv molecules are shown in FIG. 1A at panels B and C. Two IgG molecules covalently coupled (IgG2) are shown in FIG. 1A at panel D. An IgG that is specific for a disease-causing agent covalently attached to a β-1,6-linked glucans containing carbohydrate such as curdlan (c).

FIG. 1B provides a schematic overview of the mechanism of action for the removal and degradation of disease-causing agent through phagocytosis by monocytes/macrophages. The present disclosure describes the development of a molecule, such as a bispecific antibody, that binds to the phagocytic receptor Dectin-1 on one arm and a disease-causing agent (e.g. tumor cells, bacteria, viruses, LDL, protein aggregates, etc.) on the other arm. Upon engagement, the disease-causing agent is engulfed by the phagocyte and eliminated through the endo-lysosomal pathway. This may lead in the significant reduction of the disease-causing agent in tissues and blood.

FIG. 2 shows flow cytometry analysis of Dectin-1 in two healthy donor peripheral blood mononuclear cell (PBMC) samples. Single, live monocyte and lymphocyte populations were gated using fluorophore-conjugated lineage- and cell type-specific antibodies to identify respective immune cell populations. Dectin-1 expression was determined by comparing the fluorescence signal from the Dectin-1 antibody (clone 15e2; as labeled) to fluorescence minus one (FMO) and an isotype control (IgG2a; as labeled). Dectin-1 receptor number and percent of Dectin-1 positive cells (in parenthesis) are displayed in the histograms, if detected. Dectin-1 expression was detected in monocytes but not in lymphocyte populations.

FIG. 3 shows flow cytometry analysis of Dectin-1 in three healthy donor peripheral blood leukocyte (PBL) samples. Single, live monocyte and granulocyte populations were gated using forward and side scatter. Dectin-1 expression was determined by comparing the fluorescence signal from the Dectin-1 antibody (clone 15e2; as labeled) to fluorescence minus one (FMO) and isotype control (IgG2a; as labeled). Dectin-1 receptor number and percent of Dectin-1 positive cells (in parenthesis) are displayed in the histograms. Dectin-1 was expressed at lower levels in granulocytes, another class of phagocytic cells, when compared to monocytes.

FIG. 4 shows flow cytometry analysis of Dectin-1 in monocyte-derived cultured macrophages from healthy donors. Monocytes were cultured in MCSF (20 ng/ml) for 7 days to allow them to differentiate to macrophages. Single and live cells were then stained with CD11b to confirm macrophage differentiation. Dectin-1 expression was determined by comparing the fluorescence signal from the Dectin-1 antibody (clone 15e2; right peak in histograms) to fluorescence minus one (FMO) and isotype control (IgG2a; left peak in histograms). Dectin-1 expression was found to be maintained in monocyte-derived macrophages.

FIG. 5 shows flow cytometry analysis of Dectin-1 in lung immune cells from a healthy donor. Tissue lung sample from a healthy donor was dissociated using a Miltenyi Biotec tissue dissociation kit. Hematopoietic cells were gated using CD45. Lymphocyte populations were identified on CD45+ cells by using CD3+ (T cells), CD3-CD19+ (B cells), and CD3-CD56+ (NK cells) gates. Macrophages were gated using CD163 and CD11b, after excluding T, B and NK cells on CD45+ cells. Dectin-1 expression was determined by comparing the fluorescence signal from the Dectin-1 antibody (clone 15e2; right peak in histograms) to fluorescence minus one (FMO) and isotype control (IgG2a; left peak in histograms). Dectin-1 receptor number and percent of Dectin-1 positive cells (in parenthesis) are displayed in the histograms. Dectin-1 was found to be selectively expressed on macrophages but not detected in lymphocytes or in non-hematopoietic cells in healthy human lung tissue.

FIG. 6 shows flow cytometry analysis of Dectin-1 expression in control HEK293 cells (HEK-Blue Null1 Cells), HEK293 cells engineered to overexpress human Dectin-1 isoform A (HEK-Blue hDectin-1a cells) or isoform B (HEK-Blue hDectin-1b cells) and Freestyle293 cells transiently transfected with a construct expressing human Dectin-1A (293F hDectin-1a FL). Dectin-1 was detected with a Dectin-1 antibody (clone 15e2; right peak in histograms) and compared to an isotype control (IgG2a; left peak in histograms). The Dectin-1 antibody (clone 15e2) recognizes both the A and B isoforms of Dectin-1 in HEK293 cells overexpressing Dectin-1. Expression of Dectin-1 was confirmed with multiple Dectin-1 antibody clones (259931, GE2 and BD6, which only recognizes the A isoform).

FIG. 7 shows a binding analysis of Dectin-1 antibody clones 15e2 and 259931 in cynomolgus monkey monocytes derived from PBMC by flow cytometry. Single, live and CD14+ cells were gated to identify monocytes. The cells were incubated with Dectin-1 primary antibodies (clones 15e2 and 259931) and their respective isotype controls, IgG2a and IgG2b, followed by a fluorescent anti-mouse secondary antibody. The primary antibodies 15e2 and 259921 were used at a serial dose titration of 100, 33.3, 11.1, 3.7, 1.23 and 0.41 nM and the isotype controls at a serial dose titration of 166, 55.3, 18.4 and 6.150 nM. The human ectin1 antibodies (clones 15e2 and 259931) exhibited cross-reactivity to Cynomolgus Dectin-1 expressed on monocytes.

FIG. 8 shows a secreted alkaline phosphatase (SEAP) reporter assay of Dectin-1 in HEK-Blue hDectin-1a cells. HEK-Blue hDectin-1a cells were engineered to express genes in the Dectin-1/NF-kB/SEAP signaling pathway, and have a SEAP response in response to Dectin-1 ligands. SEAP production was monitored in cells incubated with Dectin-1 or isotype antibodies. Induction of alkaline phosphatase secretion by stimulation with Dectin-1 antibodies but not isotype control antibodies is shown in the upper panel of FIG. 8. The activity in cells stimulated by Dectin-1 antibodies is comparable to stimulation of HEK-Blue hDectin-1a cells with zymosan, a natural ligand of Dectin-1. The lower panel of FIG. 8 shows a dose-dependent effect of the Dectin-1 antibody on alkaline phosphatase secretion. Cells were incubated with Dectin-1 or isotype antibodies in quantities ranging from 0.1-10 μg per well to generate a dose-response curve.

FIGS. 9A & 9B show the phagocytosis of pHrodo-labeled polystyrene anti-mouse Fe IgG beads conjugated with Dectin-1 antibody or isotype control antibody by HEK-Blue hDectin-1a cells. Polystyrene anti-mouse Fe IgG beads (˜3.4 μm) were labeled with a pH-sensitive fluorescent dye (pHrodo Red) and conjugated with Dectin-1 antibody or isotype control. The beads were then incubated with cultured HEK-Blue hDectin-1a cells (50,000 per well) at a cell:beads ratio of 1:3. HEK-Blue hDectin-1a cells were labeled with the cell-permeant dye Calcein AM. The phagocytosis of the beads was monitored by IncuCyte live cell imaging or flow cytometry. Phagocytosis of the beads was quantified by the IncuCyte analysis software and expressed as overlap of pHrodo-labelled objects to calcein-positive cells. The upper panel of FIG. 9A shows the measurement of phagocytosis of the beads for over 3 hours, while the lower panel of FIG. 9A shows representative images of pHrodo positive cells at 2.5-hour time point of phagocytosis. Dectin-1 antibody coupled to beads promotes phagocytosis in HEK-Blue hDectin-1a cells. In FIG. 9B, flow cytometry measurements of phagocytosis are shown. Phagocytosis with beads coupled to Dectin-1 antibody clones (clones 15e2 and 259931) or an isotype antibody was tested. Engulfed beads are represented by the right peak in the histograms. The beads coupled to Dectin-1 antibodies induced a significantly higher level (2.1-4.5 times) of phagocytosis than the beads coupled to isotype antibody (p<0.0001; two-way anova with Holm-Sidak multiple comparison).

FIG. 10 shows the specificity of phagocytosis to Dectin-1 in HEK-Blue hDectin-1a cells. Polystyrene anti-mouse Fe IgG beads (˜3.4 μm, 400,000 per well) were labeled with pHrodo and mixed with increased amounts of Dectin-1 antibody (clone 15e2) or isotype control (IgG2a) ranging from 20 ng to 400 ng. Due to the antibody binding capacity of the beads, amounts higher than 20 ng of 15e2 antibody resulted in excess of unbound 15e2 antibody. HEK-Blue hDectin-1a cells (50,000 per well) were mixed with the Dectin-1-conjugated beads without removing unbound Dectin-1 antibody. The phagocytosis of the beads was monitored by IncuCyte live cell imaging. The phagocytosis was quantified by the IncuCyte analysis software and expressed as overlap of pHrodo-positive objects to calcein-positive cells. FIG. 10 shows the measurement of phagocytosis of beads over 4 hours (upper panel). Significant differences in phagocytosis were observed between Dectin-1 antibody amounts of 20 ng or 40 ng vs 100 ng and 100 ng vs 200 ng or 400 ng at the 4 hour time point (****p<0.0001; two-way anova with Holm-Sidak multiple comparison). The phagocytosis induced by beads coupled to Dectin-1-specific antibodies was decreased in the presence of excess amounts of free Dectin-1 antibody. FIG. 10 also shows representative images of pHrodo positive cells at the 2-hour time point of phagocytosis (lower panels).

FIGS. 11A & 11B show the phagocytosis of pHrodo-labeled polystyrene anti-mouse Fe IgG beads of different sizes conjugated with Dectin-1 antibody or isotype control antibody by HEK-Blue hDectin-1a cells. Polystyrene anti-mouse Fe IgG beads (0.85, 3.4 and 8 μm) were labeled with pHrodo Red and conjugated with Dectin-1 antibody or isotype control. The beads were then incubated with cultured HEK-Blue hDectin-1a cells (50,000 per well) at a cell:beads ratio of 1:12.) The phagocytosis of the beads was monitored by IncuCyte live cell imaging. The phagocytosis was quantified by the IncuCyte analysis software and expressed as overlap of pHrodo-positive objects to calcein-positive cells. FIG. 11A shows the phagocytosis of beads over the course of 5 hours. The Phagocytosis of beads conjugated to Dectin-1 antibody was significantly higher than beads conjugated to isotype antibody for all bead sizes tested (****p<0.0001; *p<0.05; Two-way anova with Holm-Sidak multiple comparison). FIG. 11B shows representative images of pHrodo positive cells are shown at the 5-hour time point. In the images, the arrowheads mark beads, while the circles mark pHrodo positive cells.

FIGS. 12A & 12B show the phagocytosis of pHrodo-labeled polystyrene anti-mouse Fc IgG beads conjugated with Dectin-1 antibody or isotype control antibody by HEK-Blue hDectin-1a and HEK-Blue hDectin-1b cells. Polystyrene anti-mouse Fc IgG beads (˜3.4 μm) were labeled with pHrodo Red and conjugated with Dectin-1 antibodies (clones 15e2 or 259931) or an isotype control. The beads were then incubated with cultured HEK-Blue hDectin-1a (FIG. 12A) or HEK-Blue hDectin-1b cells (FIG. 12B) (50,000 per well) at a cell:beads ratio of 1:10. Cells were labeled with the cell-permeant dye Calcein AM. The phagocytosis of the beads was monitored by IncuCyte live cell imaging over 3 hours, quantified by the IncuCyte analysis software and expressed as overlap of pHrodo-positive objects to calcein-positive cells. Phagocytosis of beads conjugated to Dectin-1 antibodies was significantly higher than beads conjugated to isotype antibody in both HEK-Blue hDectin-1a and HEK-Blue hDectin-1b cells (****,\hat{d}\hat{d}\hat{d}\hat{d}p<0.0001; ***,\hat{d}\hat{d}\hat{d}p<0.001; two-way anova with Holm-Sidak multiple comparison). Both the Dectin-1 antibody clones promoted phagocytosis at comparable levels in cells expressing the Dectin-1 isoform A (FIG. 12A). The 259931 antibody clone promoted a higher level of phagocytosis in cells expressing the Dectin-1 isoform B than the 15e2 clone (FIG. 12B).

FIGS. 13A-13C show the phagocytosis of pHrodo-labeled polystyrene anti-mouse Fc IgG beads conjugated with Dectin-1 antibody or isotype control by HEK-Blue hDectin-1a cells. Polystyrene anti-mouse Fc IgG beads of varying sizes, 0.85 (FIG. 13A), 3.4 (FIG. 13B), and 8 m (FIG. 13C), were labeled with pHrodo and conjugated with Dectin-1 antibody (clone 15e2 or 259931) or an isotype control. The beads were then incubated with cultured HEK-Blue hDectin-1a cells (50,000 per well) at a cell:beads ratio of 1:12. The phagocytosis of the beads was monitored by IncuCyte live cell imaging over 5 hours, quantified by the IncuCyte analysis software and expressed as overlap of pHrodo-positive objects to calcein-positive cells. Both of the Dectin-1 antibody clones induced a significantly higher level of phagocytosis of beads of all sizes than the isotype controls (****,\hat{d}\hat{d}\hat{d}\hat{d}p<0.0001; \hat{d}\hat{d}p<0.01; *,\hat{d}\hat{d}p,0.05; two-way anova with Holm-Sidak multiple comparison). The 259931 clone promoted similar levels of phagocytosis of the intermediate-sized particles as compared to 15e2, but promoted more efficient phagocytosis of very small and very large particles.

FIGS. 14A-14C show the phagocytosis of pHrodo-labeled polystyrene anti-mouse Fc IgG beads conjugated with Dectin-1 antibody or isotype control antibody by human monocytes. Polystyrene anti-mouse Fe IgG beads (˜3.4 μm) were labeled with pHrodo Red and conjugated with Dectin-1 antibody or isotype control. The beads were then incubated with cultured human monocytes (50,000 per well) at a cell:beads ratio of 1:3. Monocytes were labeled with the cell-permeant dye Calcein AM. The phagocytosis of the beads was monitored by IncuCyte live cell imaging. Phagocytosis was quantified by the IncuCyte analysis software and expressed as overlap of pHrodo-positive objects to calcein-positive cells. FIG. 14A shows the measurements of phagocytosis of beads over 3 hours, while FIG. 14B shows representative images of pHrodo positive cells at 2 hours of phagocytosis. Dectin-1 antibody (clone 15e2) induced a significantly higher level of phagocytosis by monocytes than the isotype control (**** p<0.0001; ***p<0.001; ** p<0.01; two-way anova with Holm-Sidak multiple comparison). Dectin-1 promoted phagocytosis of beads by human monocytes. FIG. 14C shows the flow cytometry evaluation of phagocytosis. Engulfed beads are represented by the right peak in the histograms. The beads coupled to Dectin-1 antibodies induced a significantly higher level (1.6 times) of phagocytosis by human monocytes than the beads coupled to isotype antibody.

FIG. 15 shows the phagocytosis of pHrodo-labeled polystyrene anti-mouse Fc IgG beads conjugated with Dectin-1 antibody or isotype control antibody by human monocytes in the presence of FcgR blocking antibody. Polystyrene anti-mouse Fc IgG beads (˜3.4 μm) were labeled with pHrodo and conjugated with Dectin-1 antibody or an isotype control. The beads were then incubated with cultured human monocytes (50,000 per well) at a cell:beads ratio of 1:3 in the presence of FcgR blocking antibody to exclude FcgR mediated phagocytosis. Monocytes were labeled with the cell-permeant dye Calcein AM. Images of pHrodo-positive cells were taken at 3 hours of phagocytosis. Addition of FcgR blocking antibody did not prevent Dectin-1 antibody-induced phagocytosis (cf. upper right and lower right), indicating that Dectin-1 induces phagocytosis independently from Fcy receptors.

FIG. 16 shows the phagocytosis of pHrodo labeled polystyrene anti-mouse Fc IgG beads conjugated with Dectin-1 antibody or isotype control antibody by human monocytes treated with Cytochalasin D. Polystyrene anti-mouse Fe IgG beads (˜3.4 μm) were labeled with pHrodo Red and conjugated with Dectin-1 antibody or isotype control. The beads were then incubated with cultured human monocytes (50,000 per well) at a cell:beads ratio of 1:3 in the presence or absence of 5 μM Cytochalasin D (CytoD). Monocytes were labeled with the cell-permeant dye Calcein AM. The phagocytosis of the beads was monitored by IncuCyte live cell imaging. Phagocytosis was quantified by the IncuCyte analysis software and expressed as overlap of pHrodo-positive objects to calcein-positive cells. FIG. 16 shows the measurements of phagocytosis of beads over 3 hours (upper plot), as well as representative images of pHrodo-positive cells at 3 hours of phagocytosis (lower images). Dectin-1 antibody induced phagocytosis at significantly higher levels than the isotype control (**** p<0.0001; ***p<0.001; Two-way anova with Holm-Sidak multiple comparison). Dectin-1 antibody-dependent phagocytosis was inhibited by addition of CytoD, demonstrating that actin polymerization is required for Dectin-1-directed phagocytosis in human monocytes.

FIG. 17 shows the phagocytosis of pHrodo-labeled polystyrene anti-mouse Fc IgG beads conjugated with Dectin-1 antibody or isotype control antibody by human macrophages. Polystyrene anti-mouse Fe IgG beads (˜3.4 μm) were labeled with pHrodo Red and conjugated with a Dectin-1 antibody or isotype control. The beads were then incubated with cultured monocyte-derived macrophages (50,000 per well) at a cell:beads ratio of 1:3. Macrophages were labeled with the cell-permeant dye Calcein AM. Bead phagocytosis was monitored by IncuCyte live cell imaging. Phagocytosis was quantified by the IncuCyte analysis software and expressed as overlap of pHrodo-positive objects to calcein-positive cells. FIG. 17 shows the measurements of phagocytosis of beads over 3 hours (upper plot), as well as representative images of pHrodo-positive cells at 3 hours of phagocytosis (lower images). Dectin-1 antibody induced phagocytosis by macrophages at significantly higher levels than the isotype control (**** p<0.0001; Two-way anova with Holm-Sidak multiple comparison). Dectin-1 antibody promotes directed phagocytosis of beads in cultured human macrophages.

FIGS. 18A-18C show engulfment of virus mediated by Dectin-1 bispecific antibody. Biotinylated Dectin-1 antibody (15e2-B) or biotinylated isotype (IgG2a-B) was conjugated with pHrodo-labeled streptavidin-12CA5 antibody (12CA5-SA-pHr), an anti-H3N2 antibody that binds to the hemagglutinin protein of H3N2 influenza virus. HEK-Blue hDectin-1a cells were labeled with the cell-permeant dye Calcein AM and seeded in 96-well plates (50,000 per well). The 15e2-B or isotype control were mixed with 12CA5-SA-pHrand formation of the bispecific antibodies was allowed for 30 minutes. The soluble bispecific antibodies were added to the cells at a final concentration of 40 nM. Engulfment of the 15e2-B/12CA5-SA-pHr bispecific antibody was monitored by assessing pHrodo activation with IncuCyte live cell imaging. FIG. 18A shows conjugation of the bispecific Dectin-1/12CA5 antibody to the cells. This format can be used to connect a cell with the H3N2 virus. FIG. 18B shows representative images of pHrodo positive cells at 18 hours of the experiment (engulfed 12CA5 pHrodo labelled antibody fluoresce brightly red in phagosomes). FIG. 18C shows engulfment of 15e2-B/12CA5-SA-pHr bispecific antibody over 24 hours. Engulfment was quantified by the IncuCyte analysis software and expressed as overlap of red object count (pHrodo) to calcein-positive cells. **** p<0.0001 vs isotype. Two-way anova with Holm-Sidak multiple comparison test.

FIGS. 19A & 19B show engulfment of Dectin-1 bispecific antibody by human monocytes. Biotinylated Dectin-1 antibody (15e2-B) or biotinylated isotype (IgG2a-B) was conjugated with pHrodo labeled streptavidin-12CA5 (12CA5-SA-pHr), an anti-H3N2 antibody that binds to the hemagglutinin protein of H3N2 influenza virus. Human monocytes were labeled with the cell-permeant dye Calcein AM and seeded in 96-well plates (50,000 per well). The 15e2-B or isotype control antibody was mixed with 12CA5-SA-pHr, and formation of the bispecific antibodies was allowed for 30 minutes. The soluble bispecific antibodies were added to the cells at a final concentration of 40 nM. Engulfment of the 15e2-B/12CA5-SA-pHr bispecific antibody was monitored by assessing pHrodo activation with IncuCyte live cell imaging. FIG. 19A shows engulfment of 15e2-B/12CA5-SA-pHr bispecific antibody over 21 hours, quantified by the IncuCyte analysis software and expressed as overlap of red object count (pHrodo) to calcein-positive cells. ** p<0.01; **** p<0.0001 vs isotype. Two-way anova with Holm-Sidak multiple comparison test. FIG. 19B shows representative images of pHrodo positive cells at 6 hours of the experiment (engulfed 12CA5 pHrodo labelled antibody fluoresce brightly red in phagosomes).

FIGS. 20A & 20B show engulfment of streptavidin FITC-labeled polystyrene beads (40 nm) conjugated with biotinylated Dectin-1 antibody (15e2-B) or biotinylated isotype (IgG2a-B) by human monocytes. Polystyrene FITC beads were saturated with biotinylated Dectin-1 antibody or isotype control for 30 minutes. The antibody/bead complexes were then incubated with cultured human monocytes at a ratio of 1:6 (cells:beads). FITC staining of monocytes was monitored by IncuCyte live cell imaging. FIG. 20A shows engulfment of SA-FITC beads by monocytes over 21 hours, quantified by the IncuCyte analysis software and expressed as green (FITC positive) object count. FIG. 20B shows representative images of FITC positive cells at 15 hours of the experiments.

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” with reference to a number refers to that number plus or minus 10% of that number. The term “about” with reference to a range refers to that range minus 10% of its lowest value and plus 10% of its greatest value.

There are variety of accumulated and not cleared aberrant host cells such as tumor, lymphoma, dead, necrotic, apoptotic, dying, infected, damaged cells that are associated with diseases. In addition, diverse cell products such as aggregated proteins (β-amyloid plaque or Tau aggregates), lipoprotein particles, could cause a disease upon increased accumulation. Disease-causing cell may have glycoprotein, surface protein, or glycolipid typical of aberrant cells associated with a disease, disorder, or other undesirable condition. Besides the host generated agents, variety of foreign pathogens such as infectious microbes (e.g. viruses, fungus and bacteria) and the microbe generated products and debris (e.g. viral particle envelops, endotoxin) may not be well cleared in patients. The above listed abnormalities may cause illnesses such as cancer, Alzheimer disease, fibrosis, Parkinson disease, Huntington disease, HIV, Hepatitis A, B or C, sepsis etc. Many of these disorders or diseases are characterized by an accumulation of disease-causing agents in different organs in human subjects.

Provided herein are methods for altering and improving the engulfment activity, selectivity or phenotype of a host phagocyte by using a biologic.

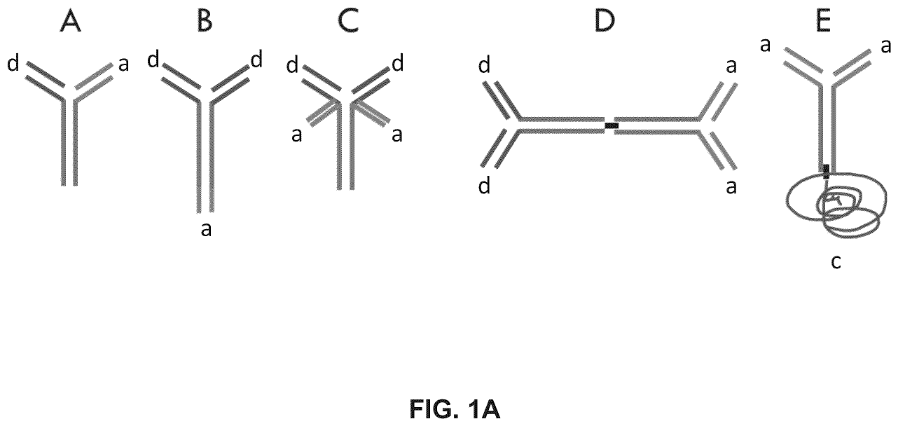

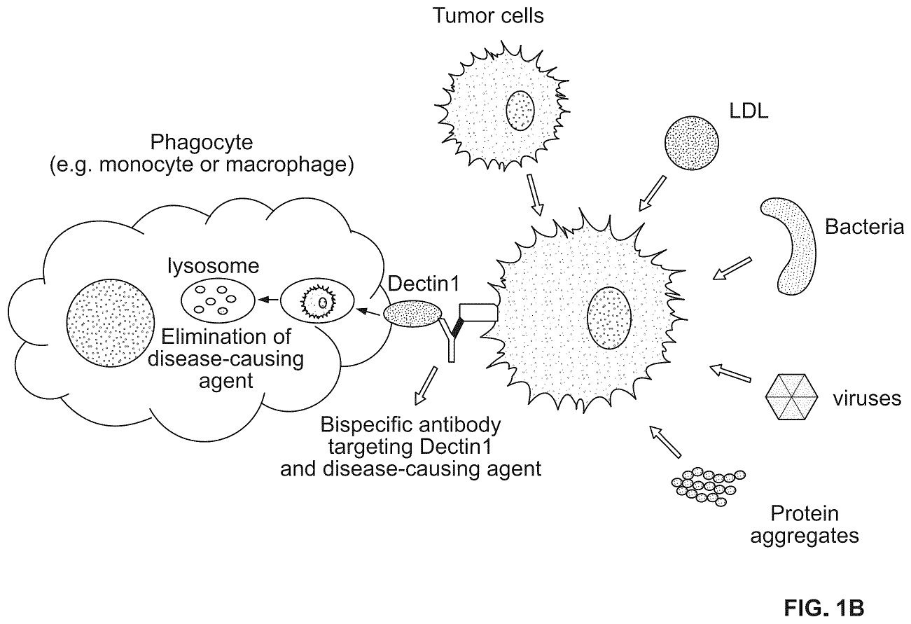

The present disclosure describes the use of molecules that specifically bind to the disease-causing agent with one arm and a phagocytotic receptor Dectin-1 receptor with the other (see, e.g., FIG. 1B). To achieve the targeted phagocytosis, it is necessary to generate a monoclonal antibody that has agonistic activity upon binding of Dectin-1. The present disclosure proposes that the agonistic antibody activates receptor and induces phagocytosis. A bispecific antibody that binds to the phagocytic receptor Dectin-1 and to a disease-causing agent such as (3-amyloid aggregate plaque, could induce phagocytosis of the agent and its degradation (FIG. 1A). In addition or alternatively to a traditional bispecific antibody, two IgGs (IgG2) covalently linked where one IgG binds to a phagocytosis receptor and the other binds to a disease causing agent could be used (FIG. 1A). Another option is to use IgG-scFv format where the IgG binds to a phagocytosis receptor and the scFv part binds to a disease-causing agent (FIG. 1A).

To enable the targeted removal of a disease-causing agent via phagocytosis, an antigen-binding domain of the present disclosure may be selected from IgGs, intrabodies, peptibodies, nanobodies, single domain antibodies, SMTPs, and multispecific antibodies (e.g., bispecific antibodies, diabodies, triabodies, tetrabodies, tandem di-scFV, tandem tri-scFv, ADAPTIR).

Multispecific antibodies have binding specificities for at least two different epitopes, usually from different antigens. Bispecific antibodies can be prepared as full length antibodies or antibody fragments (e.g. F(ab′)₂ bispecific antibodies).

Methods for making bispecific antibodies are known in the art. One well-established approach for making bispecific antibodies is the “knobs-into-holes” or “protuberance-into-cavity” approach. See e.g., U.S. Pat. No. 5,731,168. Two immunoglobulin polypeptides (e.g., heavy chain polypeptides) each comprise an interface; an interface of one immunoglobulin polypeptide interacts with a corresponding interface on the other immunoglobulin polypeptide, thereby allowing the two immunoglobulin polypeptides to associate. In some embodiments, interfaces may be engineered such that a “knob” or “protuberance” located in the interface of one immunoglobulin polypeptide corresponds with a “hole” or “cavity” located in the interface of the other immunoglobulin polypeptide. In some embodiments, a knob may be constructed by replacing a small amino acid side chain with a larger side chain. In some embodiments, a hole may be constructed by replacing a large amino acid side chain with a smaller side chain. Knobs or holes may exist in the original interface, or they may be introduced synthetically. Polynucleotides encoding modified immunoglobulin polypeptides with one or more corresponding knob- or hole-forming mutations may be expressed and purified using standard recombinant techniques and cell systems known in the art. See, e.g., U.S. Pat. Nos. 5,731,168; 5,807,706; 5,821,333; 7,642,228; 7,695,936; 8,216,805; U.S. Pub. No. 2013/0089553; and Spiess et al., Nature Biotechnology 31: 753-758, 2013. Modified immunoglobulin polypeptides may be produced using prokaryotic host cells, such as E. coli, or eukaryotic host cells, such as CHO cells. Corresponding knob- and hole-bearing immunoglobulin polypeptides may be expressed in host cells in co-culture and purified together as a heteromultimer, or they may be expressed in single cultures, separately purified, and assembled in vitro.

According to a different approach, antibody variable domains with the desired binding specificities (antibody-antigen combining sites) are fused to immunoglobulin constant domain sequences.

Bispecific antibodies include cross-linked or “heteroconjugate” antibodies. Techniques for generating bispecific antibodies from antibody fragments have also been described in the literature. For example, bispecific antibodies can be prepared using chemical linkage.

A binding protein of the present disclosure (e.g., a monoclonal antibody or antigen-binding portion) thereof may be non-human, chimeric, humanized, or human. Examples of antibody fragments include, but are not limited to, Fab, Fab′, F(ab′)2, and Fv fragments, Fab′-SH, F(ab′)2, diabodies, linear antibodies, scFv antibodies, VH, and multispecific antibodies formed from antibody fragments.

A “Fab” (fragment antigen binding) is a portion of an antibody that binds to antigens and includes the variable region and CHI of the heavy chain linked to the light chain via an inter-chain disulfide bond. An antibody may be of any class or subclass, including IgG and subclasses thereof (IgG1, IgG2, IgG3, IgG4), IgM, IgE, IgA, and IgD.

An anti-disease-causing agent antibody can be covalently attached to a phagocytosis receptor ligand such as β-1,6-linked glucans (e.g. curdlan and dextran) induces phagocytosis of the agent (FIG. 1A).

Binding of the molecule that mediates targeted removal of a disease-causing agent via phagocytosis could be with and without avidity i.e. with and without inducing dimerization of the phagocytosis receptor such as Dectin-1 or the target antigen present on the agent.

In addition to the beneficial removal of a disease-causing agent via phagocytosis, the molecule may induce production of inflammatory mediators to alter the disease microenviroment such as in tumors, cancers and lymphomas.

An immunoglobulin Fc part of the molecule that causes targeted phagocytosis may have important role in the process by engaging Fc receptors and inducing additional phagocytosis. In some embodiments, the molecule has a modified Fc domain that has reduced ADCC activity as compared to a wild type human IgG1.

Without wishing to be bound to theory, it is thought that antibody candidates with higher agonistic activity to induce phagocytosis may be the most attractive for drug development. Antibody candidates that induce low internalization may demonstrate the most pronounced phagocytosis due to the higher receptor occupancy and higher level of the receptor-antibody complex on the cell surface.

To generate monoclonal antibodies (mAb) against Dectin-1, the recombinant target will be utilized for immunization of mice. The generated mAbs will be analyzed for selective binding to Dectin-1 by ELISA and flow cytometry. The selected mAbs will be tested in vitro for Dectin-1 induced activation (phagocytosis) and internalization capabilities. The mAb candidates will be further tested for binding to cynomolgus and mouse Dectin-1. Positive candidates will be used for phagocytosis in vitro and in vivo. Activity of the selected mAbs will be compared to the commercially available mAbs. For instance, anti Dectin-1 mAbs will be tested together with the following anti Dectin-1 mAbs: 259931 (R&D Systems; Catalog #: MAB1859), 15E2 (Invitrogen Catalog #: 50-9856-42; BioLegend Catalog #: 355402), BD6 (Bio-Rad Catalog #: MCA4662), GE2 (Abcam Catalog #: Ab82888); REA515 (Miltenyi Biotec Catalog #: 130-107-725).

To generate monoclonal antibodies (mAb) against the disease-causing agent such as β-amyloid aggregate, the agents will be utilized for immunization of mice. The generated mAbs will be analyzed for selective binding to the appropriate target by ELISA or flow cytometry is applicable. The mAb candidates with the highest affinity will be further tested for binding to cynomolgus targets if applicable. Phagocytotic activity of the anti Dectin-1 mAb candidate will be compared to a commercially available anti Dectin-1 mAbs. Positive candidates will be used to produce a bispecific antibody comprised of an arm that binds to Dectin-1 and to a disease-causing agent such as β-amyloid aggregate.

Antibodies may be produced using recombinant methods. For example, nucleic acid encoding the antibody can be isolated and inserted into a replicable vector for further cloning or for expression. DNA encoding the antibody may be readily isolated and sequenced using conventional procedures (e.g., via oligonucleotide probes capable of binding specifically to genes encoding the heavy and light chains of the antibody). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody can be produced intracellularly, in the periplasmic space, or directly secreted into the medium. If the antibody is produced intracellularly, the particulate debris, either host cells or lysed fragments, are removed, for example, by centrifugation or ultrafiltration. Where the antibody is secreted into the medium, supernatants from such expression systems are generally first concentrated using a commercially available protein concentration filter.

The final candidates will be used for phagocytosis in vitro and in vivo. Activity of the selected candidates will be compared to the ligand induced phagocytosis as reported (Herre 2004).

To demonstrate in vitro phagocytotic activity of the final candidates for depletion of a disease-causing agent, an in vitro model which recapitulates activity in humans is used. Peripheral blood lymphocytes (PBL) isolated from normal blood donors will be incubated with the final candidates to study the depletion. Level of the agent will be measured by ELISA or flow cytometry. Phagocytotic activity of the antibodies will be tested with purified primary monocytes as previously described (Ackerman 2011). To demonstrate phagocytotic activity of the candidates on macrophages we will produce them from primary monocytes. In addition, the Ab activity on primary tissue cells comprised of macrophages and DCs from single cell tissue homogenates as well as bone marrow or synovial fluid is studied.

To show activity of the selected antibody candidates in vivo for depletion or reduction in levels of the disease-causing agents such as LDL or E. coli, mice or cynomolgus monkeys will be used. A cohort of cynomolgus monkeys will be bled one day prior to the single dose antibodies treatment to identify the pre dose level of LDL by ELISA. Upon treatment with antibodies, the monkeys will be bled at the following timepoints: 1 hour, 1, 7, 14 and 30 days. Level of a disease-causing agents such as LDL in blood and other biospecimens such as synovial fluids, bone marrow and spleen will be determined by ELISA.

The final mAb candidate will be human or humanized and characterized for binding to human and cynomolgus phagocytotic receptor Dectin-1, and disease-causing agent such as LDL, phagocytosis abilities, and in vivo activity. In addition, the final candidate needs to be soluble at concentrations higher than 10 mg/mL, has low level of soluble aggregates (<5%), maintains its binding to the targets as measured by ELISA (>90% potency), with no degradation products as measured by SDS PAGE when incubated for 3 months at 2-8° C.

Toxicology analysis of the final humanized candidate will be performed in cynomolgus monkeys at doses that are more than 5 times higher than the doses anticipated to be used in human subjects.

Without wishing to be bound to theory, it is thought that the molecule that performs targeted phagocytosis may demonstrate clear benefits for patients such as Alzheimer disease, Parkinson disease, cancer, infectious diseases (viral, bacterial, fungal, protozoan infections), inflammatory, or immune diseases (e.g., autoimmune diseases, inflammatory bowel diseases, multiple sclerosis), degenerative disease (e.g., joint and cartilage) Rheumatoid arthritis, Felty's syndrome, aggressive NK leukemia, IBM, IBD etc. In addition, targeted phagocytosis antibody treatment may have better activity of depleting cells in tissues over ADCC that relies on NK cells. The treatment may have a selective activity for removal of a particular disease-causing agent over a therapy that targets myeloid cells and improves phagocytosis in general.

Accordingly, the present disclosure provides, inter alia, a method of reducing the number or depleting of disease-causing agents in a human subject upon administration of molecule that induces targeted phagocytosis by binding to a phagocytotic receptor and the agent and has an immunoglobulin Fc region.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Examples

### Example 1: Analysis of Dectin-1 Expression

This Example describes the results of experiments to characterize expression of Dectin-1 by various cell types.

### Materials and Methods

### Healthy Donor Samples

Fresh healthy donor buffy coats were obtained from Stanford Blood Center. Peripheral blood mononuclear cells (PBMCs) were isolated via ficoll-paque (GE Healthcare, Chicago, IL) separation and cryopreserved in Bambanker cell freezing media (Bulldog-Bio, Portsmouth, NH). Briefly, buffy coats were diluted in phosphate buffered saline (PBS) in 1:1 ratio, followed by layering of the diluted buffy coat in ficoll and centrifugation at 760 g. The PBMC layer was isolated and washed in PBS prior to downstream analysis. Peripheral blood leukocytes (PBLs) were isolated through red blood cell lysis. Tissue samples were provided by the Cooperative Human Tissue Network which is funded by the National Cancer Institute. Tissue dissociation was performed according to the manufacturer's instructions of the Miltenyi Biotec tumor dissociation kit (Miltenyi Biotec Inc., Auburn, CA). Cryopreserved cynomolgus monkey PBMC were obtained from Human Cells.

### Primary Cells and Cell Culture

Human monocytes were isolated from healthy donor PBMCs according to the manufacturer's instructions of the pan-monocyte isolation kit (Miltenyi Biotec Inc., Auburn, CA). For macrophage differentiation, monocytes from PBMCs were let to attach on cell culture plates for 3 hours. The floating cells were washed off and the attached monocytes were cultured in 20 ng/ml MCSF (Peprotech, Rocky Hill, NJ) for 7 days to fully differentiate into macrophages. HEK-Blue Null1 Cells (Invivogen, San Diego, CA) were maintained in DMEM/10% FBS supplemented with Normocin and Zeocin. HEK-Blue hDectin-1a cells and HEK-Blue hDectin-1b cells (Invivogen, San Diego, CA) were maintained in DMEM/10% FBS supplemented with Normocin and Puromycin.

Freestyle 293-F cells were transiently transfected according to the manufacturer's suggestion (Thermo Fisher, Waltham, MA). Briefly, viable cell density and percent viability was determined. Cells were diluted to a final density of 1×10*6 viable cells/mL with Freestyle 293 Expression Medium. Freestyle Max Reagent was diluted with OptiPro SFM Medium, mixed and incubated at room temperature for 5 minutes. The diluted Freestyle Max Reagent was added to plasmid DNA diluted with OptiPro SFM Medium and mixed. The Freestyle Max Reagent/plasmid DNA complexes were incubated at room temperature for 10-20 minutes. The complexes were slowly transferred to the cells, swirling the culture flask gently during the addition, and the cells were then incubated in a 37° C. incubator with >80% relative humidity and 8% CO2 on an orbital shaker.

### Flow Cytometry Analysis

Approximately 1×10⁵-5×10⁵ cells were plated in non-tissue culture treated, 96-well V bottom plates and incubated in human FcgR blocking antibody (Biolegend, San Diego, CA) for 10 minutes at room temperature. The cells were subsequently stained with the eFluor 506 viability dye (ThermoFisher, Waltham, MA) in 1:1000 dilution for 30 minutes on ice, followed by a wash step in FACS buffer (PBS with 2% fetal bovine serum). An antibody cocktail was added to the cells, then incubated on ice for 30 minutes, followed by another wash step in FACS buffer. Ultracomp beads (ThermoFisher, Waltham, MA) were used for antibody compensation. The antibodies used in this study are provided in Table 1. All data acquisition and fluorescence compensation were performed using a CytoFlex flow cytometer (Beckman Coulter, Atlanta, GA). Data analysis was performed using the FlowJo flow cytometry data analysis software. The strategy used for determining Dectin-1 expression in monocytes, lymphocytes and granulocytes was by gating individually on forward and side scatter. Single cells were gated using forward scatter area and forward scatter height, followed by live cell gating using eFluor 506 and forward scatter area. Monocyte, T cell, B cell, NK cells and granulocytes were gated using CD14, CD3+/CD4+/8+, CD3-CD19+, CD3-CD56+ and CD15+ markers, respectively. Cultured macrophages were identified by CD11b staining. In lung tissue, hematopoietic cells were gated using CD45. T cell, B cell and NK cells were gated on CD45+ cells using CD3+, CD3-CD19+, CD3-CD56+ strategies respectively. Macrophages were gated using CD163 and CD11b, after excluding T, B and NK cells on CD45+ cells. For binding assays, primary Dectin-1 antibodies were used at a titration of 100, 33.3, 11.1, 3.7, 1.23 and 0.41 nM and the isotype controls at a titration of 166, 55.3, 18.4 and 6.150 nM followed by a fluorescently-labeled anti-mouse Fc-specific secondary antibody.

### Receptor Quantification

Dectin-1 receptor number was quantified by staining healthy donor PBMCs with APC-conjugated target antibodies and gated based on the appropriate immune cell types as described above. Quantum APC molecules of equivalent soluble fluorochrome (MESF) calibration standard beads (Bangs Laboratories, Inc., Fishers, IN) were acquired and analyzed concurrently to allow conversion of median fluorescence intensity measurements to MESF units, according to the manufacturer's protocol. Background fluorescence was removed by subtracting the FMO (fluorescence minus one) and isotype control MESF values. MESF values were subsequently divided by the fluorophore to protein ratio (provided by the manufacturer) to convert to antibody binding capacity or receptor number.

### Antibodies

Table 1 provides the antibodies used in the experiments described in the Examples.

TABLE 1
Fluorescently-labeled antibodies.

| | | | Catalog | | |
|---|---|---|---|---|---|
| Target | Clone | Fluorophore | number | Vendor | Dilution |
| Dectin-1 | 15e2 | — | 355402 | Biolegend | |
| Dectin-1 | 15e2 | APC | 355406 | Biolegend | 1:67 |
| Dectin-1 | 259931 | — | MAB1859 | R&D Systems | |
| Dectin-1 | 259931 | APC | FAB17561A | R&D Systems | 1:20 |
| Dectin-1 | BD6 | — | MCA4662GA | Biorad | |
| Dectin-1 | BD6 | Alexa Fluor 647 | MCA4662A647 | Biorad | 1:20 |
| Dectin-1 | GE2 | — | MA5-16692 | ThermoFisher | |
| CD11b | ICRF44 | Pacific blue | 301315 | Biolegend | 1:20 |
| CD14 | HCD14 | FITC | 325604 | Biolegend | 1:20 |
| CD14 | HCD14 | PE-Cy7 | 368606 | Biolegend | 1:20 |
| CD3 | SK7 | Pacific blue | 344824 | Biolegend | 1:20 |
| CD4 | OKT4 | Alexa Fluor 700 | 317426 | Biolegend | 1:40 |
| CD8 | SK1 | PerCP-Cy5.5 | 344710 | Biolegend | 1:20 |
| CD19 | B4 | Brilliant violet | 302244 | Biolegend | 1:67 |
| | | 605 | | | |
| CD16 | 3G8 | APC-Cy7 | 557758 | BD Bioscience | 1:200 |
| CD56 | B159 | FITC | 562794 | BD Bioscience | 1:67 |
| CD45 | HI30 | BV650 | 304044 | Biolegend | 1:40 |
| CD163 | GHI/61 | APC-Cy7 | 333622 | Biolegend | 1:40 |
| HA | 12CA5 | — | RT0268 | bioxcell | |
| mIgG1 | MOPC-21 | APC | 400120 | Biolegend | — |
| mIgG2a | MOPC-21 | APC | 981906 | Biolegend | — |
| mIgG2b | 27-35 | APC | 402206 | Biolegend | — |
| mIgG2a | MOPC-173 | — | 400224 | Biolegend | — |
| mIgG2b | 27-35 | — | 402202 | Biolegend | — |
| mIgG1 | MG3-35 | — | 401302 | Biolegend | |

### Results

The expression of Dectin-1, also known as CLEC7A, in various cell types was evaluated using Dectin-1-specific antibodies and flow cytometry analysis. Single, live monocyte and lymphocyte populations from donor samples or cultured cell samples were analyzed by flow cytometry, using fluorophore-conjugated lineage- and cell type-specific antibodies to identify respective immune cell populations. Dectin-1 was detected using a Dectin-1-specific antibody. Dectin-1 expression was determined by comparing to fluorescence minus one (FMO) and isotype control antibody. In some experiments, Dectin-1 receptor number and percent of Dectin-1 positive cells were calculated. All antibodies used in Dectin-1 detection and flow cytometry are listed in Table 1.

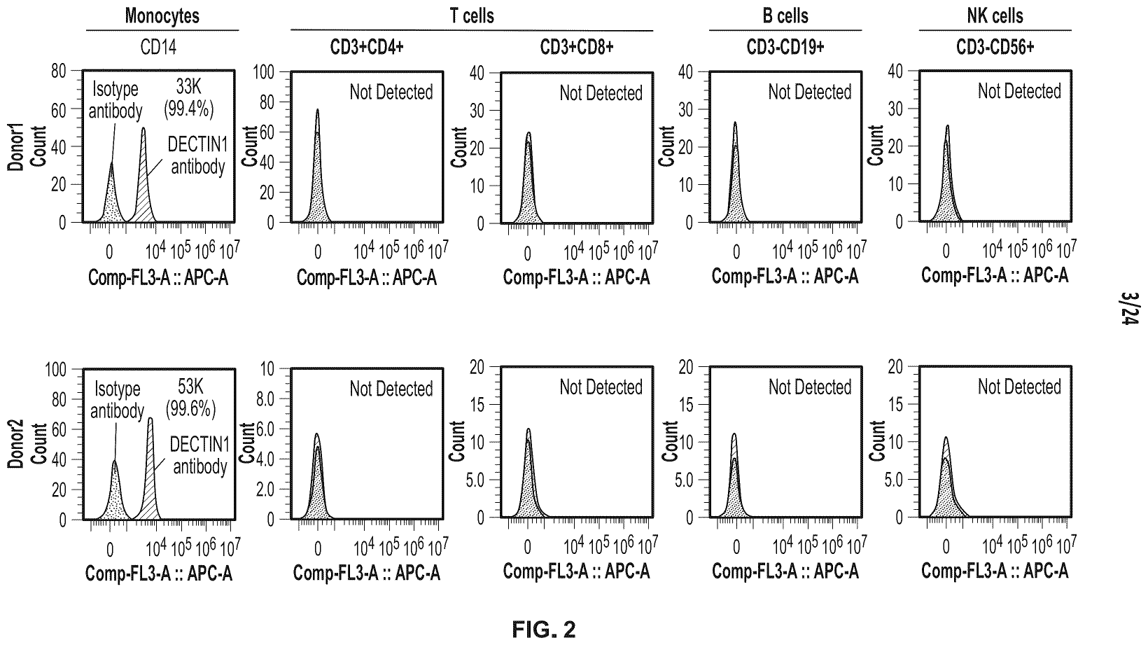

To determine the expression of Dectin-1 in immune cell populations, two healthy donor peripheral blood mononuclear cell (PBMC) samples were collected and analyzed by flow cytometry. A high level of Dectin-1 expression was found on monocytes (CD14+ cells) of healthy PBMC samples (FIG. 2). Monocytes are professional phagocytic cells. Expression of Dectin-1 in monocytes was positive in 21 of 22 donors tested, and the percentage of Dectin-1 positive monocytes was over 90% with receptor number ranging from 32,000 to 59,000 per cell. Dectin-1 was not detected on CD4+ T-cells (CD3+CD4+ cells), CD8 T-cells (CD3+CD8+ cells), B cells (CD3-CD19+ cells), or NK cells (CD3-CD56+ cells). Thus, Dectin-1 is selectively expressed on monocytes and not on T cells, B cells or NK cells in healthy donor PBMC samples.

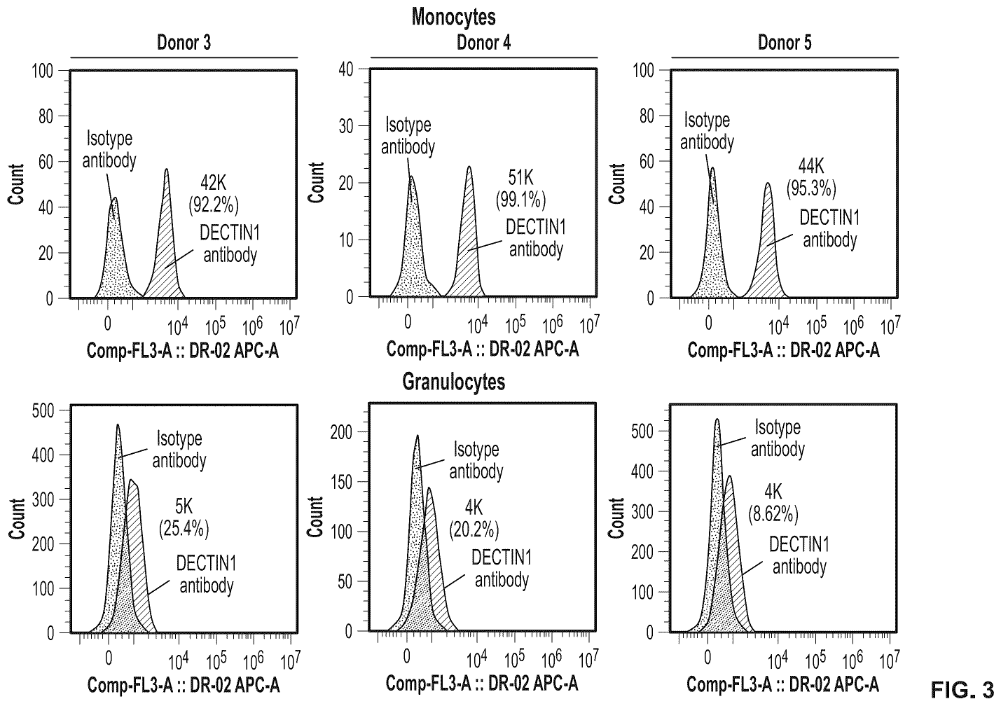

As Dectin-1 is highly expressed on monocytes, the expression of Dectin-1 in granulocytes was also examined. Granulocytes are another type of phagocytic immune cells. Three healthy donor peripheral blood leukocyte (PBL) samples were collected and analyzed by flow cytometry. As shown in FIG. 3, Dectin-1 was highly expressed on monocytes and modestly expressed in granulocytes in three healthy donor PBL samples. Dectin-1 was expressed in granulocytes at lower levels compared to monocytes, with receptor number from 4,000 to 5,000 per cell.

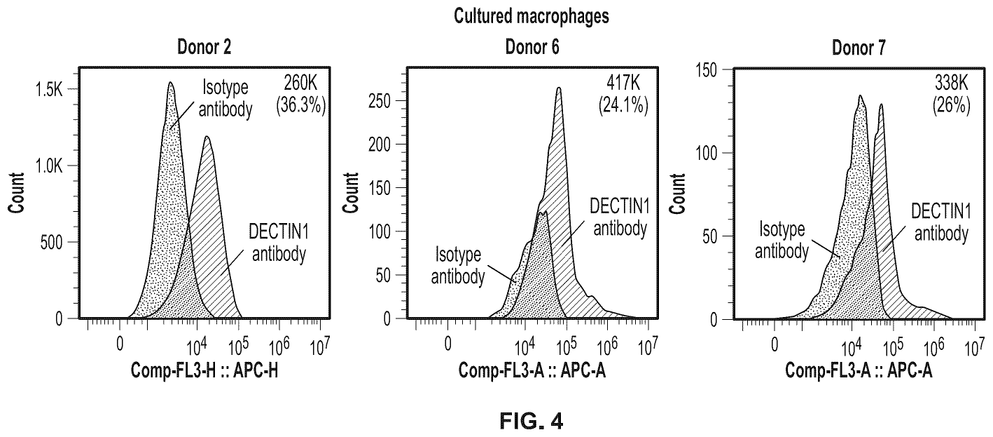

Monocytes can differentiate into macrophages, which are tissue-specific phagocytic cells. To determine the expression of Dectin-1 on macrophages, donor samples were cultured in MCSF (20 ng/ml) for 7 days to allow them to differentiate to macrophages. Single and live cells were then stained with CD11b to confirm macrophage differentiation, then analyzed by flow cytometry to determine Dectin-1 expression. As shown in FIG. 4, Dectin-1 is expressed on monocyte-derived cultured macrophages. The confirmation that Dectin-1 expression is retained in cultured monocyte-derived macrophages served as proof-of-principle that targeted phagocytosis is possible in tissues.

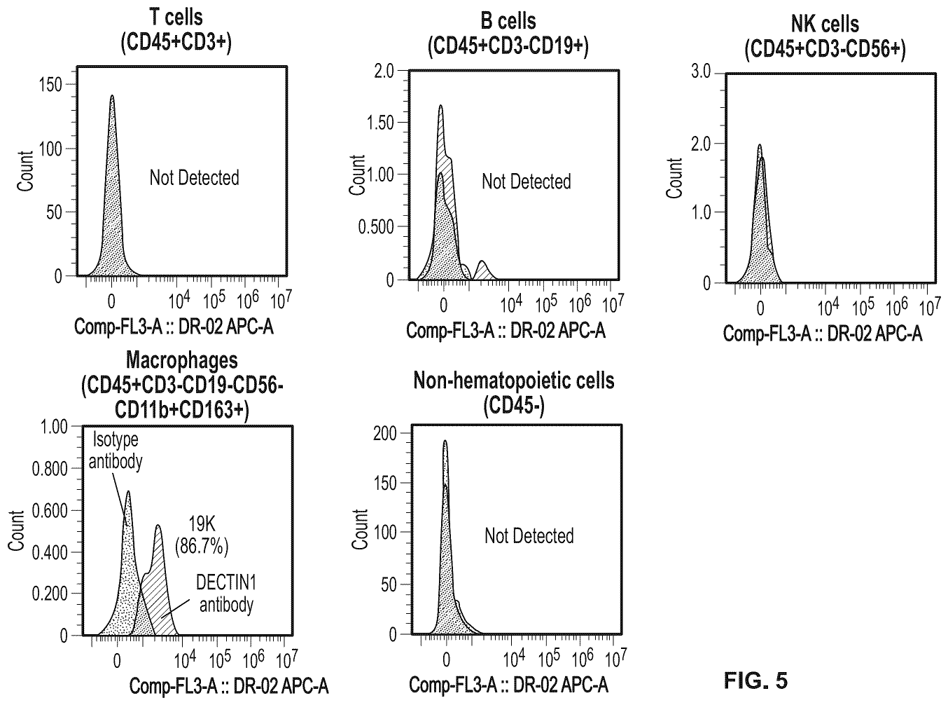

Macrophages are tissue-specific phagocytic cells. To test the expression of Dectin-1 in macrophages within a tissue, a lung tissue sample from a healthy donor was collected, dissociated, and analyzed by flow cytometry. Hematopoietic cells were gated using CD45 to separate them from non-hematopoietic cells in the tissue. T cell, B cell and NK cells were identified on CD45+ cells using CD3+, CD3-CD19+, CD3-CD56+ gates, respectively. Macrophages were gated using CD163 and CDllb, after excluding T, B, and NK cells on CD45+ cells. Dectin-1 expression was determined for all isolated cell populations. FIG. 5 shows the results of this experiment. Dectin-1 was highly expressed in macrophages in lung tissue sample, with receptor numbers of 19,000 per cell. Dectin-1 expression was not detected in T cells, B cells, or NK cells, indicating that Dectin-1 is selectively expressed in macrophages in healthy human lung tissue. Dectin-1 was not detected in non-hematopoietic cells. This result demonstrates that Dectin-1-mediated targeted phagocytosis in tissues is possible, as both the appropriate cell type and the target are present.

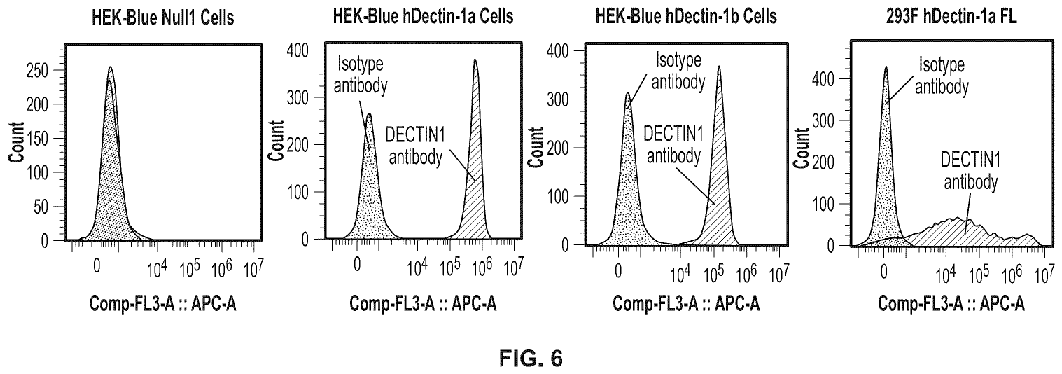

The Dectin-1 receptor can be expressed as two different isoforms, isoform A and isoform B. To examine whether the Dectin-1 antibodies recognized either the A or B isoforms, HEK293 cells were engineered to overexpress human Dectin-1 isoform A or B (HEK-Blue hDectin-1a cells and HEK-Blue hDectin-1b cells, respectively), and analyzed by flow cytometry to assess Dectin-1 expression. The 15e2 Dectin-1 antibody clone was used to confirm Dectin-1 expression. Control HEK293 cells (HEK-Blue Null1 cells) and Freestyle293 cells transiently transfected with a construct expressing human Dectin-1A (293F hDectin-1a FL) were analyzed to test the specificity of Dectin-1 detection. The 15e2 Dectin-1 antibody cloned recognized both the A and B isoforms of Dectin-1 in HEK293 cells overexpressing Dectin-1, as shown in FIG. 6. The antibody is specific to Dectin-1, as no Dectin-1 was detected in untransformed control cells. The engineered HEK293 cells are a useful tool for functional evaluation of phagocytosis and signaling events involving Dectin-1 in a normally non-phagocytic cell line.

The specificity of multiple Dectin-1 antibody clones (259931, GE2, and BD6) was also evaluated in HEK293 cells overexpressing Dectin-1 and in monocytes from healthy donors. The results of these experiments are summarized in Table 2. Clone 259931 had the highest affinity to Dectin-1 in all cells tested. The 259931 clone also had high affinity for both isoforms A and B of Dectin-1, while other antibodies do not bind or have diminished binding affinity for the B isoform. The different affinities observed for the different Dectin-1 antibody clones could result from binding to different epitopes, as evidenced by their differing affinities to the receptor isoforms.

TABLE 2
Affinity of Dectin-1 antibody clones in Dectin-1 overexpressing cell lines

| Dectin-1 | HEK-Blue | HEK-BLUE | HEK293F | Human |
|---|---|---|---|---|
| antibody | hDectin-1a cells | hDectin-1b cells | hDectin-1a FL | monocytes |
| clone | EC50 (nM) | EC50 (nM) | EC50 (nM) | EC50 (nM) |
| 15e2 | 1.2 | 9.3 | 1.4 | 0.6 |
| 259931 | 0.8 | 0.9 | 1.2 | 0.3 |
| GE2 | 1.9 | 12 | 2.4 | 1.4 |
| BD6 | 9.8 | — | N/A | — |

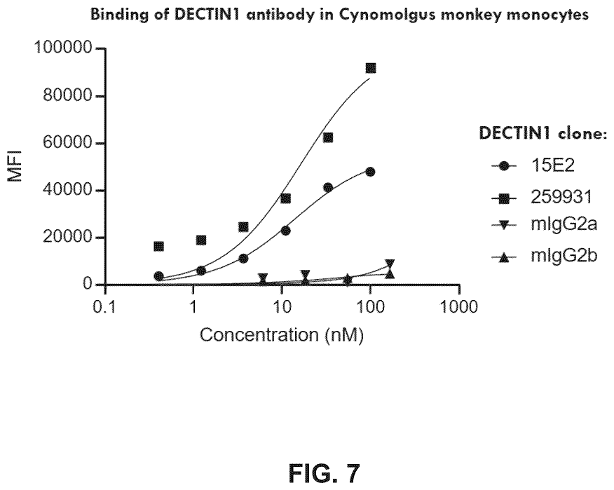

Finally, a binding assay was performed to examine the cross-reactivity of the human Dectin-1 antibody clones 15e2 and 259931 to Cynomolgus monkey Dectin-1. Monocytes were derived from monkey PBMC samples by flow cytometry. The isolated cells were incubated with either the 15e2 and 259931 Dectin-1 antibody clones, and their respective isotype control antibody, IgG2a and IgG2b, followed by a fluorescent anti-mouse secondary antibody. To generate a binding curve, the Dectin-1 antibodies were used at a serial dose titration of 100, 33.3, 11.1, 3.7, 1.23 and 0.41 nM, while the isotype controls were used at a serial dose titration of 166, 55.3, 18.4 and 6.150 nM. As shown in FIG. 7, both of the Human Dectin-1 antibody clones cross-reacted to monkey Dectin-1 expressed on monocytes. However, the clones exhibited different binding characteristics of each clone on monkey Dectin-1, which demonstrates that the different antibodies bind to different epitopes. Because cynomolgus monkeys are commonly used as a pre-clinical model for toxicological studies, and these Dectin-1 antibodies bind to cynomolgus monkey monocytes, they can therefore easily be used for toxicological studies.

As shown in this example, Dectin-1 is highly expressed in monocytes and macrophages, specialized phagocytic cells, but not in other immune cells. Dectin-1 expression is also specific to macrophages within healthy human lung tissue. The Dectin-1 antibodies characterized in this example specifically recognize Dectin-1 in cells, can recognize both isoforms of Dectin-1, and cross-react to monkey Dectin-1. The antibodies described in this example can be used for Dectin-1-mediated targeted phagocytosis.

### Example 2: Effect of Dectin-1 Antibody on Phagocytosis and Signaling

This Example describes the results of experiments to test the effects of the Dectin-1 antibody on phagocytosis and signaling.

### Materials and Methods

Materials and methods used in this experiment are detailed below. Unless otherwise noted, donor samples and primary cells were prepared as described in Example 1. Unless otherwise noted, cell culture, flow cytometry, and receptor quantification were performed as described in Example 1. Antibodies used in this example are described in Table 1.

SEAP Reporter Assay in HEK Cells with Dectin-1 Antibodies Immobilized by Air Drying

Dectin-1 monoclonal antibodies 15e2, 259931, GE2, BD6 and control isotypes were immobilized by coating onto the surfaces of wells of untreated 96-well, U-bottomed polypropylene microtiter plates. To coat, 10 g of the antibody diluted in 50 μl sterile PBS was added to each well. Plates were left overnight in a class II laminar flow cabinet with the lids removed to allow the solutions to evaporate. Coated plates were washed twice with 200 μl sterile PBS to remove salt crystals and unbound antibody. HEK-Blue hDectin-1a cells were then cultured on the plates for 22 hours and alkaline phosphatase levels were assessed in the supernatant at OD 630 nm using QUANTI-Blue Solution (Invivogen, San Diego, CA) per manufacturer's instructions.

Labelling of Polystyrene Beads with pHrodo and Conjugation to Antibodies

pHrodo labelling was performed using polystyrene beads coated with Goat anti-Mouse IgG (Fc) (Spherotech, Lake Forest, IL). The beads were washed with Phosphate Buffered Saline pH 7.2 (PBS) (Corning, Corning, NY) using a Spin-X centrifuge tube filters (Corning, Corning, NY). The pH was adjusted by addition of bicarbonate buffer. pHrodo Red, succinimidyl ester (pHrodo Red, SE) (ThermoFisher, Waltham, MA) was added to the beads and allowed to incubate for 60 minutes at room temperature with shaking. The beads were then washed with PBS using Spin-X Centrifuge Tube Filters to remove excess pHrodo RED. After pHrodo labeling, the antibody was conjugated to the beads according to the manufacturer's recommendations. Briefly, based on the binding capacity of the beads to antibody, an excess of antibody was added to the beads in PBS and allowed to incubate at room temperature for 60 minutes with shaking. The beads were then washed with PBS using Spin-X centrifuge tube filters to remove unbound antibody.

### Antibody-Dependent Cellular Phagocytosis

For phagocytosis experiments, HEK cells overexpressing Dectin-1 or monocytes were seeded in a 96-well plate and let attach for 1 hour. pHrodo beads conjugated to Dectin-1 antibodies or isotypes were added at the desired ratio. Differentiated macrophages were detached using Accutase (Thermo Fisher, Waltham, MA) and reseeded in a 96-well plate at the desired density and allowed to attach for 2 hours before adding the beads. Cell tracker Calcein AM (Thermo Fisher, Waltham, MA) was added in to identify live cells.

Plates containing cells and pHrodo-conjugated beads were placed in an IncuCyte S3 live imaging system (Sartorius, Germany). Phagocytosis was monitored by taking images at desired time points and analyzed using the IncuCyte S3 software. The overlap of bright red fluorescence (engulfed beads) with Calcein AM-positive cells was taken as a measure of phagocytosis.

In some experiments pHrodo-labelled beads were mixed with Dectin-1 antibodies in a 96-well plate for 1 hour. The beads were spun down, and the supernatant was aspirated to remove unbound antibody. Cells were then mixed with the beads at the desired ratio, briefly spun down and monitored for phagocytosis. Alternatively, cells, incubated with the beads for 30 minutes or 1 hour, were collected and phagocytosis was assessed by flow cytometry using a CytoFlex flow cytometer (Beckman Coulter, Atlanta, GA).

For bispecific antibody preparation, single antibodies were conjugated to biotin or strepatividin (Abcam, Cambridge, MA) and pHrodo label (where indicated). The antibodies were mixed at a ratio of 2:1 (biotin antibody: streptavidin antibody) and allowed to bind for 30 minutes at room temperature. The bispecific antibodies were added to cells to investigate engulfment by IncuCyte live imaging. In one experiment biotinylated antibodies were mixed with streptavidin-FITC beads, 40 nm in size (Thermo Fisher, Waltham, MA)

### Results

The Dectin-1-specific antibodies described in Example 1 were assayed for their ability to activate secretion of alkaline phosphatase and phagocytosis in various cell types. Unless otherwise noted, phagocytosis made with polystyrene anti-mouse Fe IgG beads (˜3.4 μm) labeled with a pH-sensitive fluorescent dye (pHrodo Red) and conjugated with Dectin-1 antibody or isotype control. Stimulation of Dectin-1 by ligands results in the production of secreted alkaline phosphatase (SEAP) in cells. Dectin-1-specific antibodies could act as ligands of the receptor and stimulate the SEAP signaling pathway in cells.

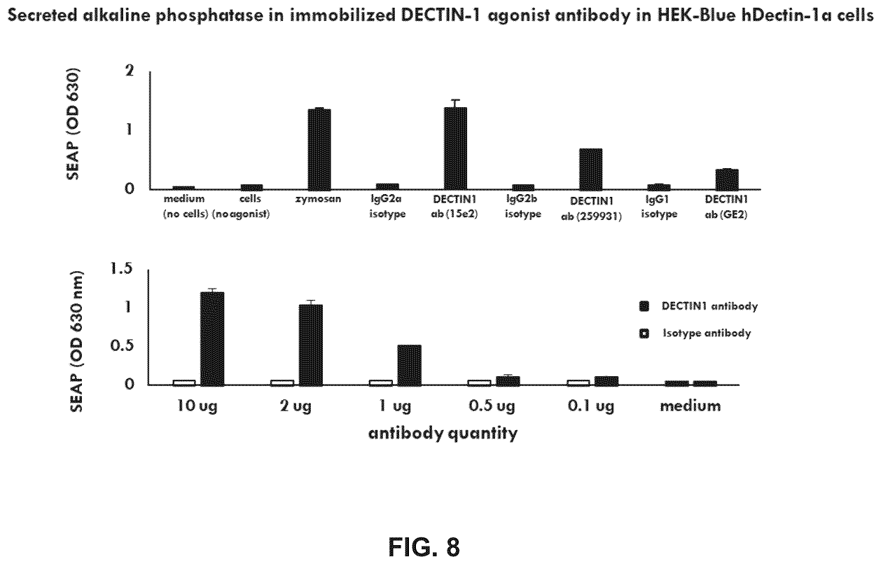

The ability of the Dectin-1 antibodies in stimulating Dectin-1 was tested by a SEAP reporter assay using HEK-Blue hDectin-1a cells. HEK-Blue hDectin-1a cells have been engineered to express Dectin-1A isoform and genes involved in the Dectin-1/NF-κB/SEAP signaling pathway and thus express a secreted alkaline phosphatase (SEAP) in response to stimulation by Dectin-1 ligands. As a positive control cells were incubated with zymosan (10 ug/ml), a natural ligand of DECTIN. As shown in FIG. 8, the 15e2 Dectin-1 antibody clone promotes SEAP secretion, likely by engaging Dectin-1 on the surface of the cells indicating an agonistic activity. The activity resulting from stimulation by the Dectin-1 antibody is comparable to zymosan. The effect of the Dectin-1 antibody is also dose-dependent, as shown in FIG. 8. Thus, Dectin-1-specific antibodies induce alkaline phosphatase secretion in HEK-Blue hDectin-1a. These cells provide a useful tool to functionally screen Dectin-1 antibodies.

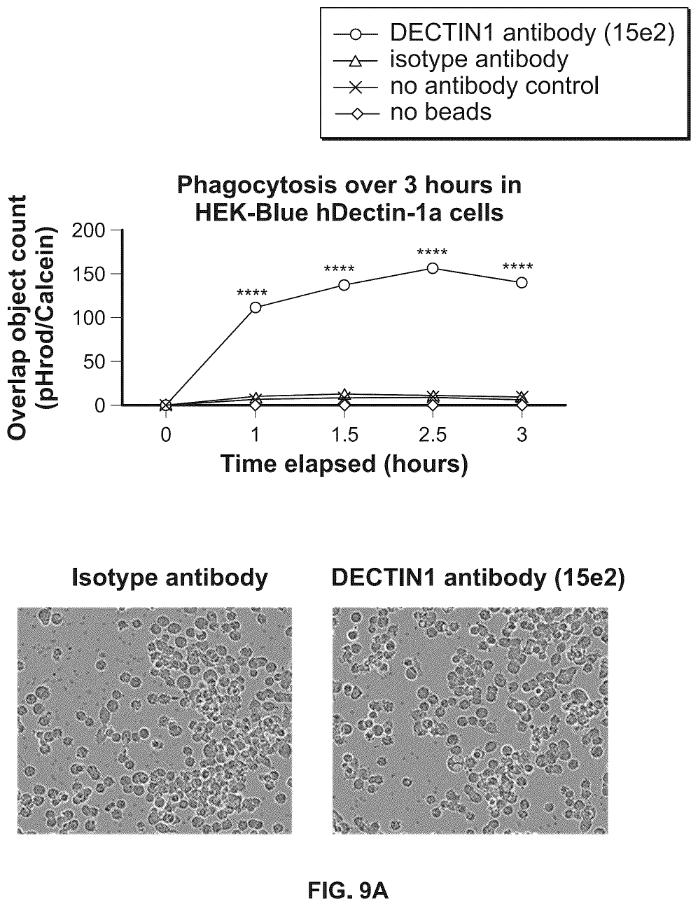

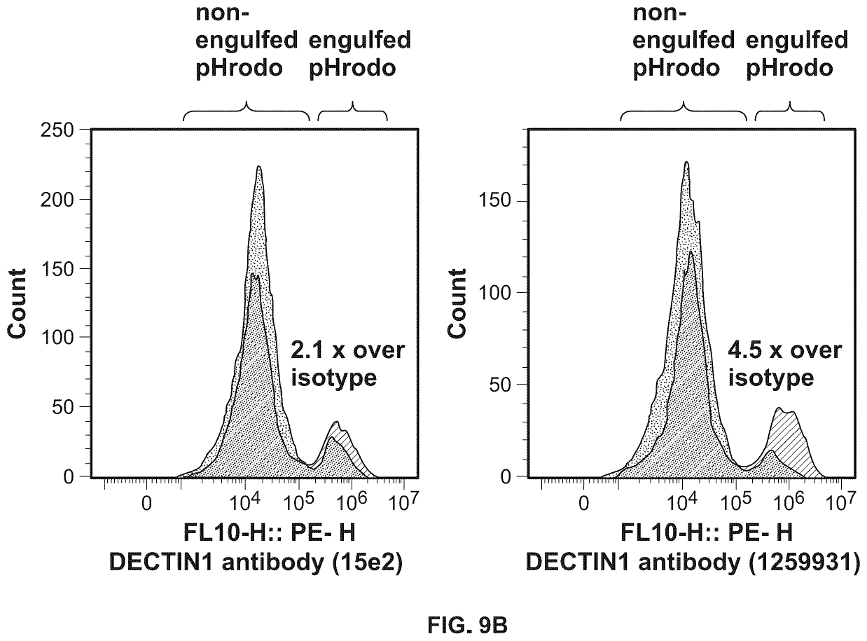

Stimulation of Dectin-1 can lead to activation of phagocytosis. To investigate Dectin-1-specific phagocytosis, cultured HEK-Blue hDectin-1a cells were treated with pHrodo-labeled beads conjugated with Dectin-1 antibody or isotype control antibody. The fluorescent signal produced by pHrodo increases in acidic environments, such as the environment found in a phagosome. As shown in FIGS. 9A & 9B, Dectin-1 antibody-coupled beads promote phagocytosis in HEK-Blue hDectin-1a cells. As shown in FIG. 9B, pHrodo-labelled beads conjugated with the 259931 Dectin-1 antibody clone promoted a higher level of phagocytosis over the isotype control (4.5 times higher than isotype control) than the 15e2 clone (2.1 times higher than isotype control). Treatment of HEK-Blue hDectin-1a was sufficient to induce targeted phagocytosis. Some cells also engulfed multiple beads, indicating a high efficiency of internalization. As HEK cells do not express Fcγ receptors, another receptor involved in phagocytosis, and are not normally phagocytic, these results are indicative of high specificity towards Dectin-1-dependent phagocytosis

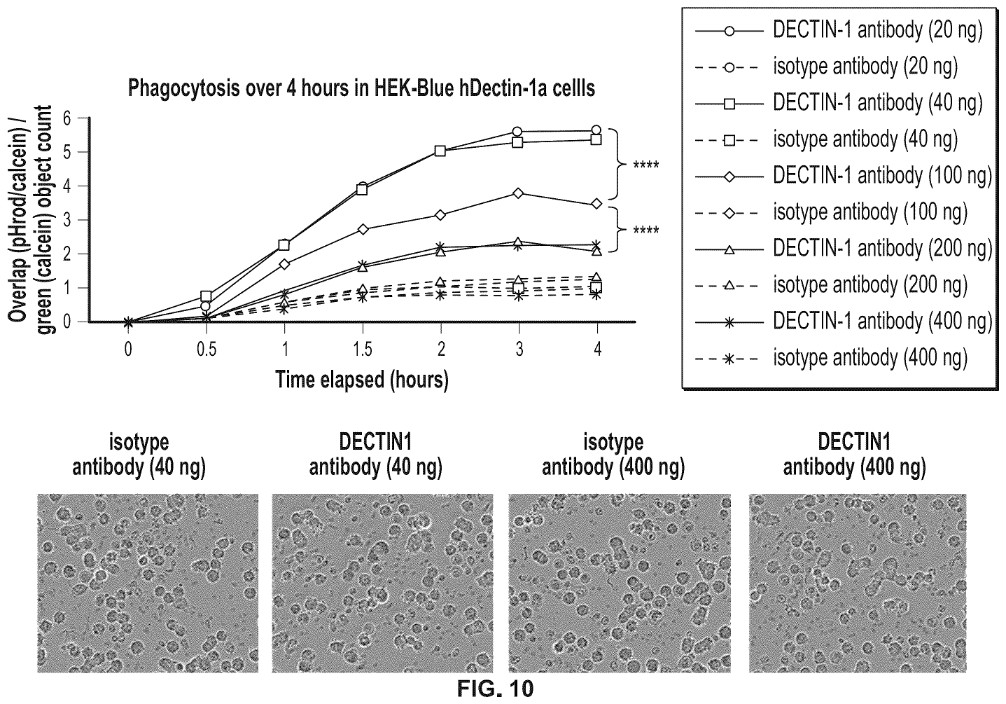

The specificity of the Dectin-1-mediated phagocytosis observed upon stimulation with the Dectin-1 antibody was further tested by a competition assay. If the observed phagocytosis is due to Dectin-1 receptor stimulation by the Dectin-1 antibody-conjugated beads, then addition of free Dectin-1 antibody is expected to decrease the phagocytosis of the beads. In this experiment, pHrodo-labelled beads were mixed with increased amounts of Dectin-1 antibody or isotype control (IgG2a) ranging from 20 ng to 400 ng. Because 20 ng of antibody are needed to occupy all binding sites of 400,000 beads (according to the manufacturer's instructions), any amount higher than 20 ng would result in excess amount of unbound antibody. As shown in FIG. 10, the level of Dectin-1-specific antibody-induced phagocytosis was decreased in the presence of excessive free Dectin-1 antibody. The excess free antibody competed with pHrodo-labelled Dectin-1 antibody-conjugated beads leading to a decrease in phagocytosis of the beads. This observation supports that antibody-induced phagocytosis is specific to Dectin-1 stimulation.

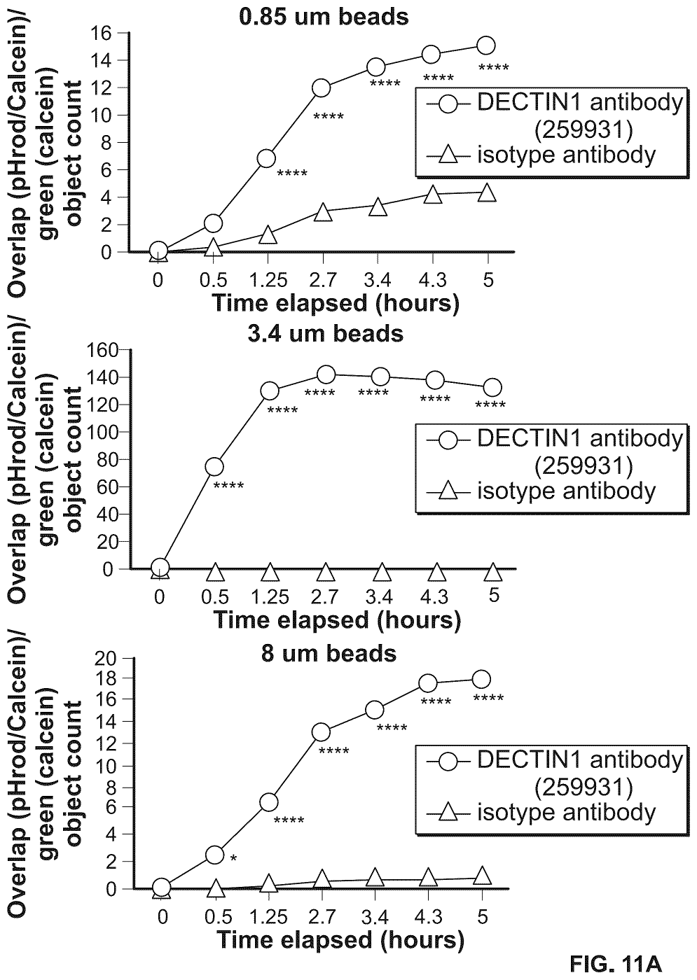

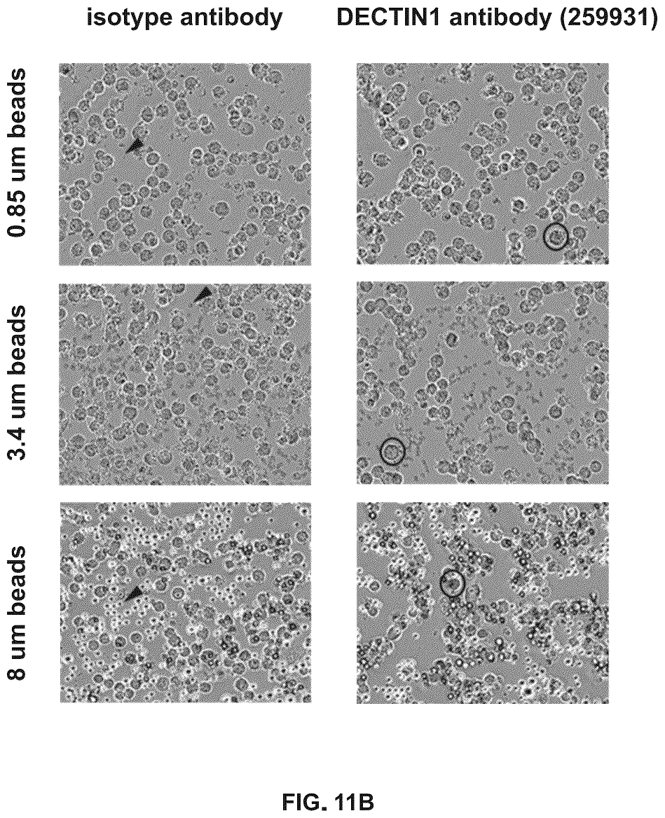

Phagocytosis was also examined using three sizes of pHrodo-labelled beads, 0.85 μm, 3.4 μm, and 8 μm, conjugated to Dectin-1 antibody or isotype control. All three sizes of beads were found to be taken up via Dectin-1-mediated phagocytosis (FIGS. 11A & 11B). These data support the conclusion that Dectin-1 can efficiently engulf particles that differ in size within the size range of disease-causing agents such as cells (˜10-20 μm), bacteria (˜0.2-2 μm), larger viruses (˜0.5-1 μm), and protein aggregates.

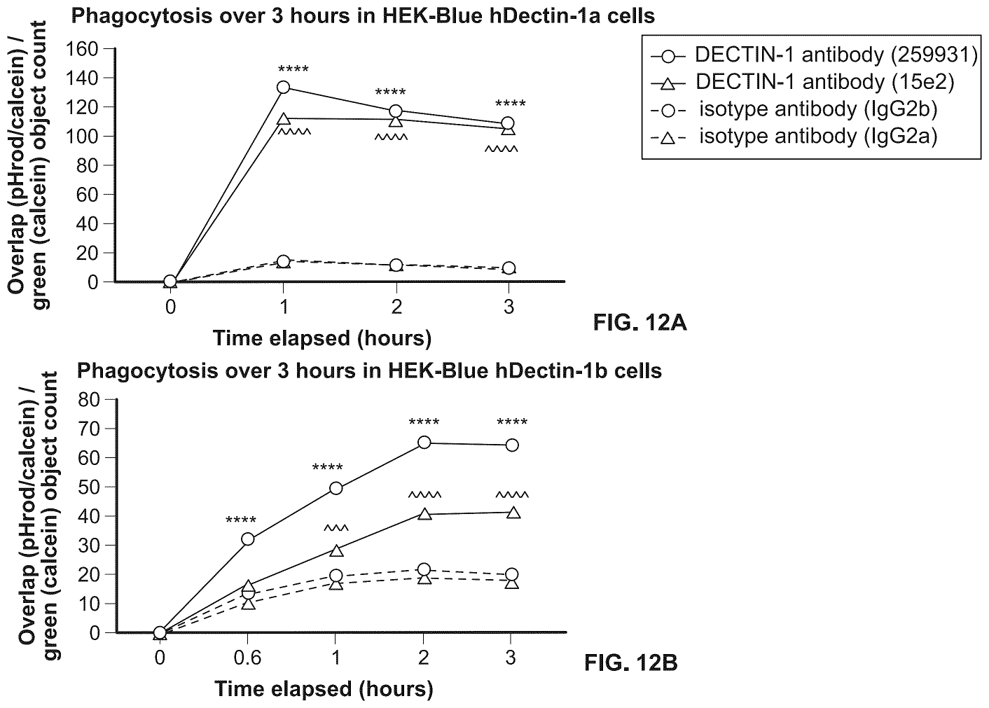

As Dectin-1 is expressed as two different isoforms, the ability of the Dectin-1 antibody to stimulate phagocytosis by both isoforms A and B of Dectin-1 was tested. HEK-Blue hDectin-1a and HEK-Blue hDectin-1b cells were incubated with pHrodo-labelled beads conjugated with Dectin-1 antibodies or isotype control. The 15e2 or 259931 Dectin-1 antibody clones conjugated to beads were tested in this experiment. These two clones can bind to both the A and B isoforms of Dectin-1 with different affinities (see Table 2). As shown in FIGS. 12A & 12B, the 15e2 and 259931 Dectin-1 antibody clones promoted phagocytosis at comparable levels in HEK cells overexpressing isoform A of Dectin-1. However, the 259931 promoted a higher level of phagocytosis in the HEK cells overexpressing isoform B of Dectin-1 than the 15e2 clone. As shown in Table 2, the 259931 clone had higher affinity for isoform B than the 15e2 clone. This result indicates that the specific epitope engaged by the Dectin-1 antibody has a differential effect on the phagocytic ability, depending on the Dectin-1 isoform that is expressed. Dectin-1 antibodies can promote phagocytosis in both Dectin-1 isoform A and B overexpressing cells lines and therefore promote phagocytosis in primary cells that express either form of Dectin-1.

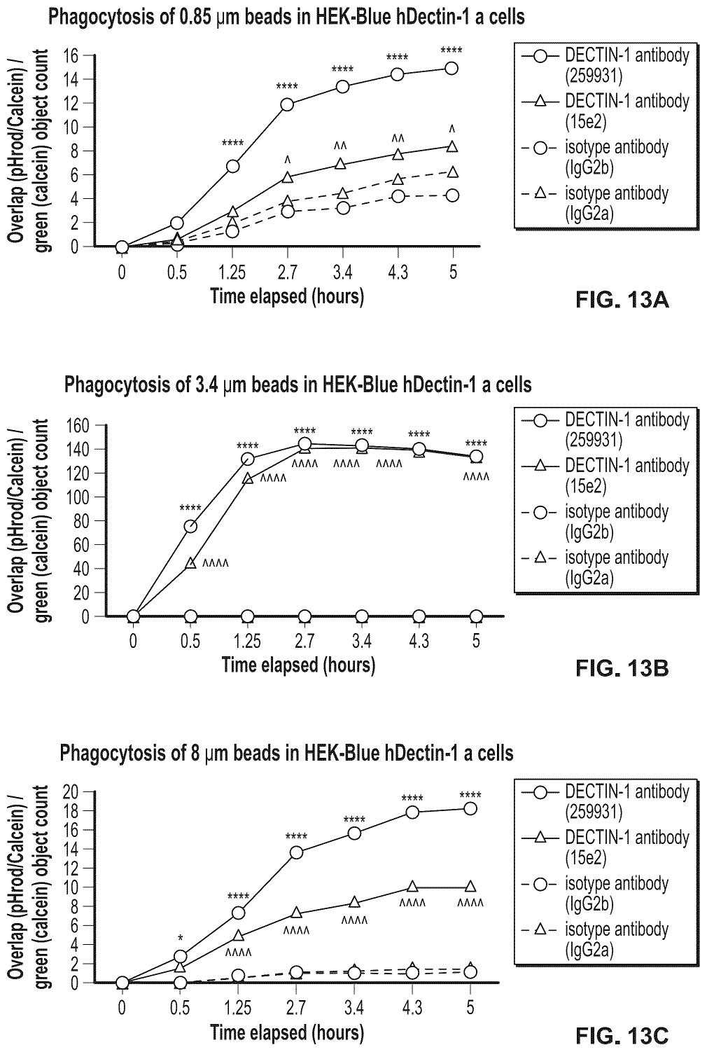

Because the 259931 Dectin-1 antibody clone performed better in promoting phagocytosis in cells expressing either the A or B isoforms of Dectin-1, the ability of this antibody clone to promote the engulfment particles of different sizes was tested. These results are shown in FIGS. 13A-13C. Both the 259931 and the 15e2 Dectin-1 antibody clones promoted phagocytosis of medium particles at comparable efficiency. However, the 259931 clone promoted phagocytosis of very small or very large particles more efficiently than the 15e2 clone. This result shows that the two Dectin-1 antibodies have different ability to ingest smaller or larger particles, indicating that the engaged epitope is associated with superior functional phagocytic ability.

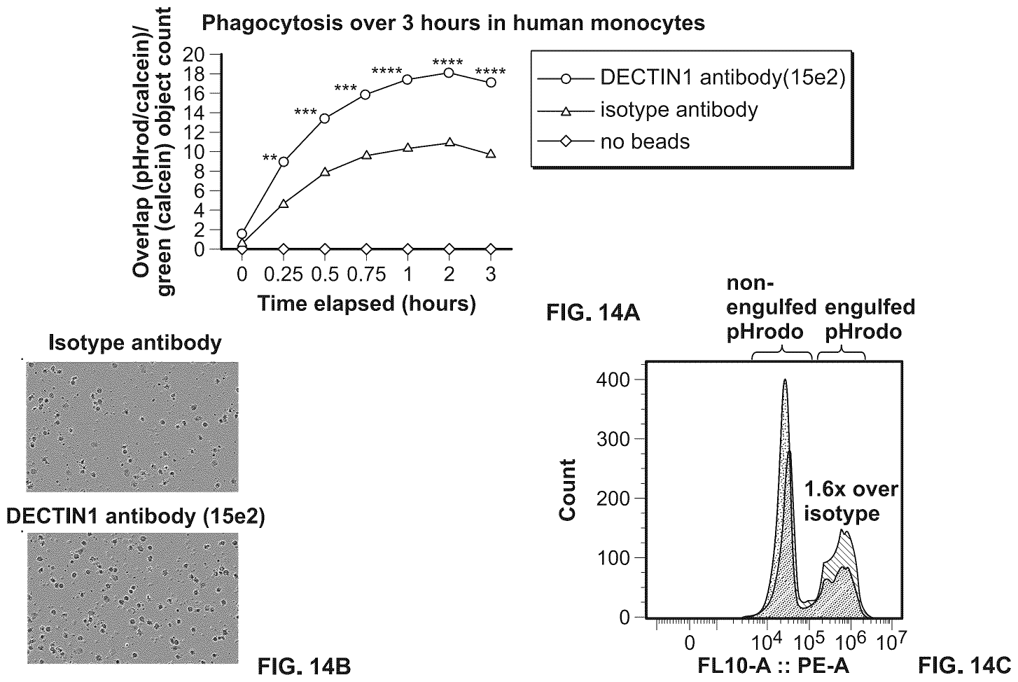

As described in Example 1, Dectin-1 is highly expressed in human monocytes, a type of phagocytic cell. To determine if phagocytosis in monocytes can be promoted by antibody engagement of Dectin-1, purified monocytes (CD14+) from human PBMC were incubated with pHrodo-labeled beads conjugated with Dectin-1 antibody. As shown in FIGS. 14A-14C, Dectin-1 antibody-conjugated beads promoted phagocytosis by monocytes at significantly higher levels (1.6 times higher) than that of isotype control beads. Thus, in addition to promoting phagocytosis in cells overexpressing Dectin-1, Dectin-1-specific antibodies promote phagocytosis in human monocytes.

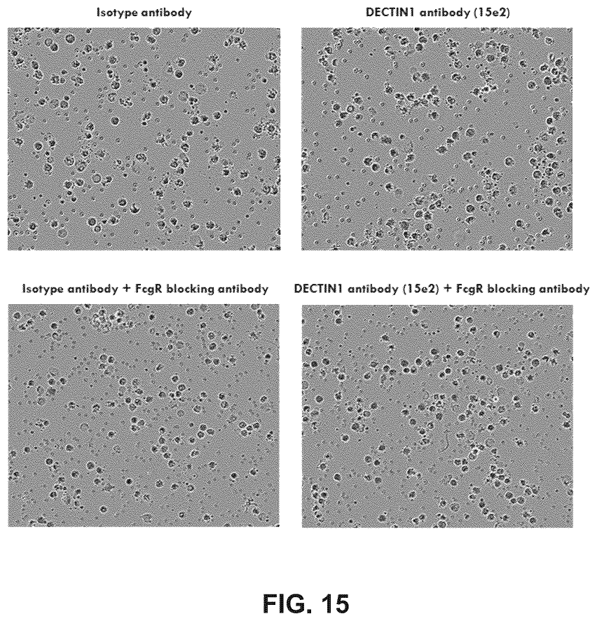

The activation of phagocytosis in monocytes is specific to stimulation of Dectin-1 and independent of Fcγ receptors (FcγRs). As shown in FIG. 15, addition of an antibody to block FcγRs did not affect the induction of phagocytosis by the Dectin-1 antibody-conjugated beads. The directed phagocytosis in monocytes is thus induced by Dectin-1 antibodies is Dectin-1-specific and not due to FcγR-mediated phagocytosis.

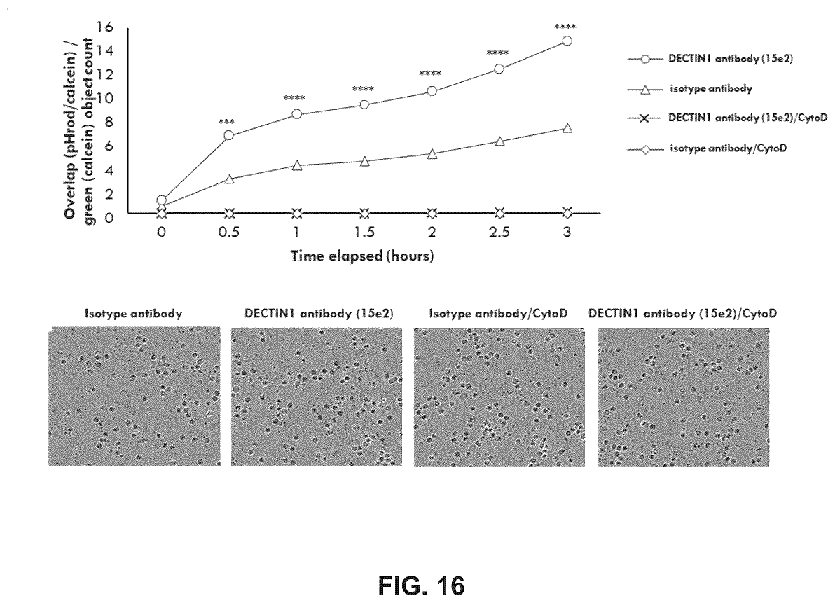

Because Dectin-1-mediated phagocytosis requires the actin cytoskeleton, the effect of addition of Cytochalasin D (CytoD), an actin depolymerizing drug, was also tested. Monocytes were incubated with Dectin-1 antibody-conjugated beads in the presence of absence of CytoD. As shown in FIG. 16, Dectin-1-mediated phagocytosis was inhibited by treatment with CytoD, demonstrating a requirement of the actin cytoskeleton. Because active actin polymerization is required for phagocytosis and Dectin-1-mediated phagocytosis was sensitive to treatment with CytoD, Dectin-1 antibody-mediated targeted phagocytosis is specific to this type of cellular transport and not through a non-specific or passive mechanism.

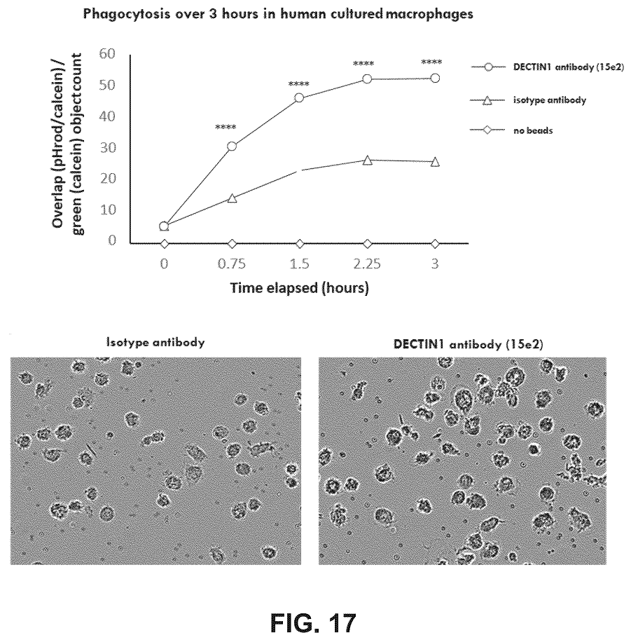

Finally, the ability of the Dectin-1 antibodies to promote phagocytosis in human macrophages was analyzed. Purified monocytes were cultured in MCSF (20 ng/ml) for 7 days to differentiate in macrophages. The monocyte-derived macrophages were then incubated with Dectin-1 antibody-conjugated beads to test for Dectin-1-mediated phagocytosis in these cells. As shown in FIG. 17, DECTIN-1 antibody promoted directed phagocytosis of beads in cultured human macrophages. A higher frequency of phagocytosis and a greater number of engulfed beads was observed in cells incubated with Dectin-1 antibody-conjugated beads than with isotype control beads. These results demonstrate that Dectin-1-mediated phagocytosis in tissues via macrophage-expressed Dectin-1 is possible.

The results presented in this example highlight that robust, targeted depletion is possible in different compartments such as blood, bone marrow and tissue.

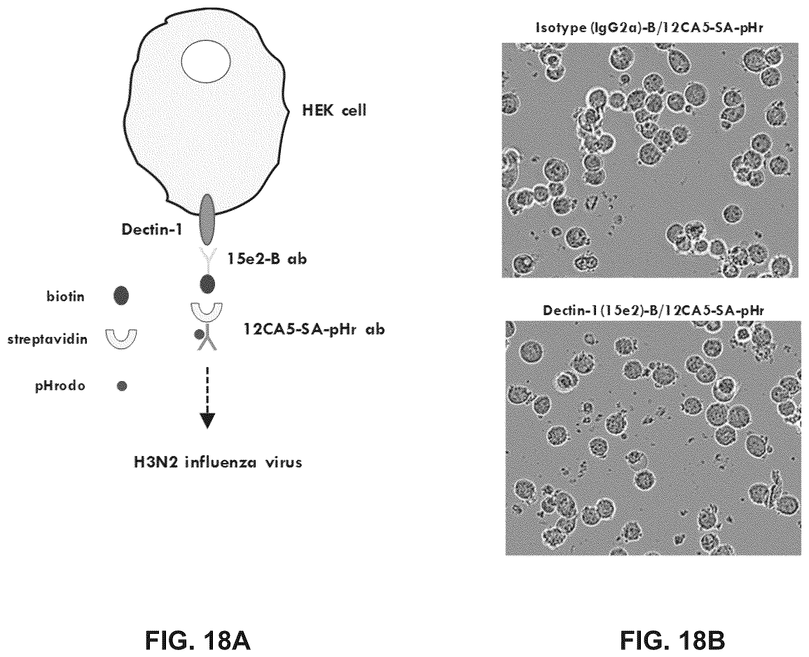

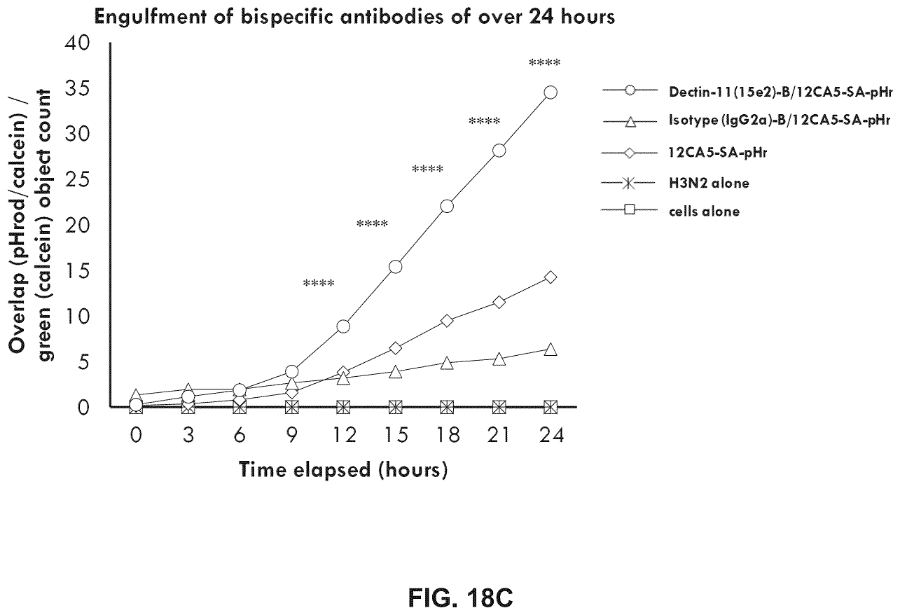

Next, engulfment of Dectin-1 antibody conjugated to a pHrodo-labeled anti-H3N2 virus antibody was examined in recombinant cell lines overexpressing Dectin-1, providing proof-of-concept that demonstrates engulfment of a virus mediated by Dectin-1 bispecific antibody. Biotinylated Dectin-1 antibody (15e2-B) or biotinylated isotype (IgG2a-B) was conjugated with pHrodo-labeled streptavidin-12CA5 antibody (12CA5-SA-pHr), an anti-H3N2 antibody that binds to the hemagglutinin protein of H3N2 influenza virus. HEK-Blue hDectin-1a cells were labeled with the cell-permeant dye Calcein AM and seeded in 96-well plates (50,000 per well). The 15e2-B or isotype control were mixed with 12CA5-SA-pHrand formation of the bispecific antibodies was allowed for 30 minutes. The soluble bispecific antibodies were added to the cells at a final concentration of 40 nM. Engulfment of the 15e2-B/12CA5-SA-pHr bispecific antibody was monitored by assessing pHrodo activation with IncuCyte live cell imaging. A diagram of conjugation of the bispecific Dectin-1/12CA5 antibody to the cells is shown in FIG. 18A. This format can be used to connect a cell with the H3N2 virus.

At 18 hours, representative images showed pHrodo positive cells (engulfed 12CA5 pHrodo labelled antibody fluoresce brightly red in phagosomes; FIG. 18B). FIG. 18C shows engulfment of 15e2-B/12CA5-SA-pHr bispecific antibody over 24 hours. Engulfment was quantified by the IncuCyte analysis software and expressed as overlap of red object count (pHrodo) to calcein-positive cells.

These results demonstrate that a bispecific antibody targeting Dectin-1 and a disease-causing agent (e.g., the H3N2 influenza virus) can cause the agent to be engulfed in HEK cells overexpressing Dectin-1. These data indicate that a bispecific antibody targeting Dectin-1 and a small biological agent, such as influenza virus (˜100 nm), could be used to connect Dectin-1-expressing cells to a disease-causing agent for engulfment and elimination via phagocytosis.

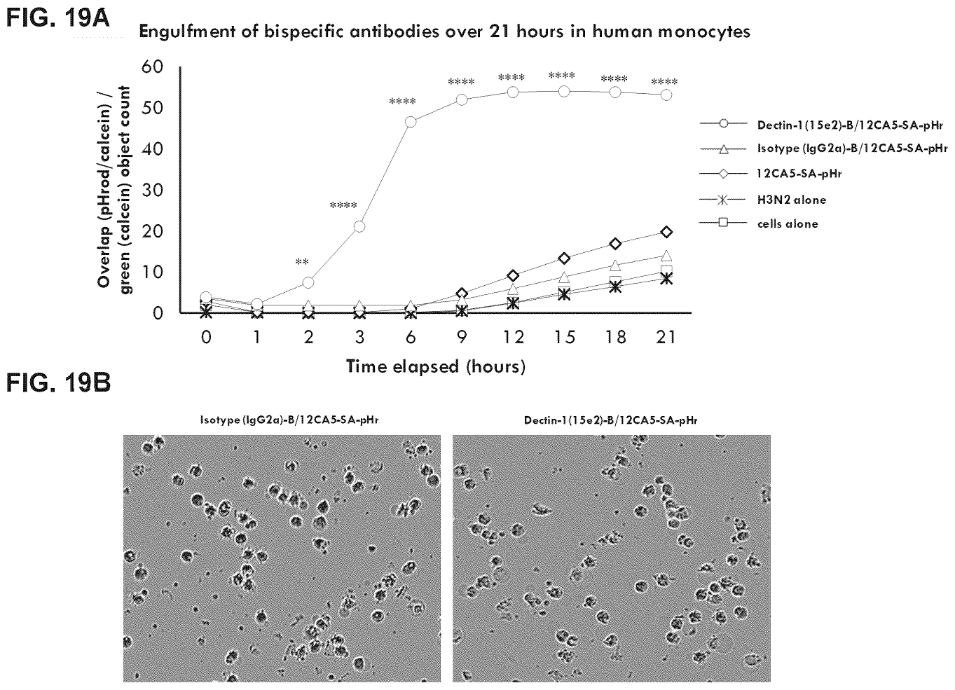

Next, engulfment of Dectin-1 antibody conjugated to a pHrodo-labeled anti-H3N2 virus antibody was examined in primary human monocytes, providing proof-of-concept that demonstrates engulfment of a virus mediated by Dectin-1 bispecific antibody in primary human phagocytic cells. FIGS. 19A & 19B show engulfment of Dectin-1 bispecific antibody by human monocytes. Biotinylated Dectin-1 antibody (15e2-B) or biotinylated isotype (IgG2a-B) was conjugated with pHrodo labeled streptavidin-12CA5 (12CA5-SA-pHr), an anti-H3N2 antibody that binds to the hemagglutinin protein of H3N2 influenza virus. Human monocytes were labeled with the cell-permeant dye Calcein AM and seeded in 96-well plates (50,000 per well). The 15e2-B or isotype control antibody was mixed with 12CA5-SA-pHr, and formation of the bispecific antibodies was allowed for 30 minutes. The soluble bispecific antibodies were added to the cells at a final concentration of 40 nM. Engulfment of the 15e2-B/12CA5-SA-pHr bispecific antibody was monitored by assessing pHrodo activation with IncuCyte live cell imaging. FIG. 19A shows engulfment of 15e2-B/12CA5-SA-pHr bispecific antibody over 21 hours, quantified by the IncuCyte analysis software and expressed as overlap of red object count (pHrodo) to calcein-positive cells. ** p<0.01; **** p<0.0001 vs isotype. Two-way anova with Holm-Sidak multiple comparison test. FIG. 19B shows representative images of pHrodo positive cells at 6 hours of the experiment (engulfed 12CA5 pHrodo labelled antibody fluoresce brightly red in phagosomes).

These results indicate that monocytes engulfed the Dectin-1/H3N2 influenza virus bispecific antibody. These data support the concept that a Dectin-1 bispecific binding protein could be utilized to promote engulfment of a disease-causing agent, such as the influenza virus, by human monocytes. This highlights the possibility of eliminating these disease-causing agents for the treatment of the infectious diseases by infusion of a soluble Dectin-1 bispecific antibody.

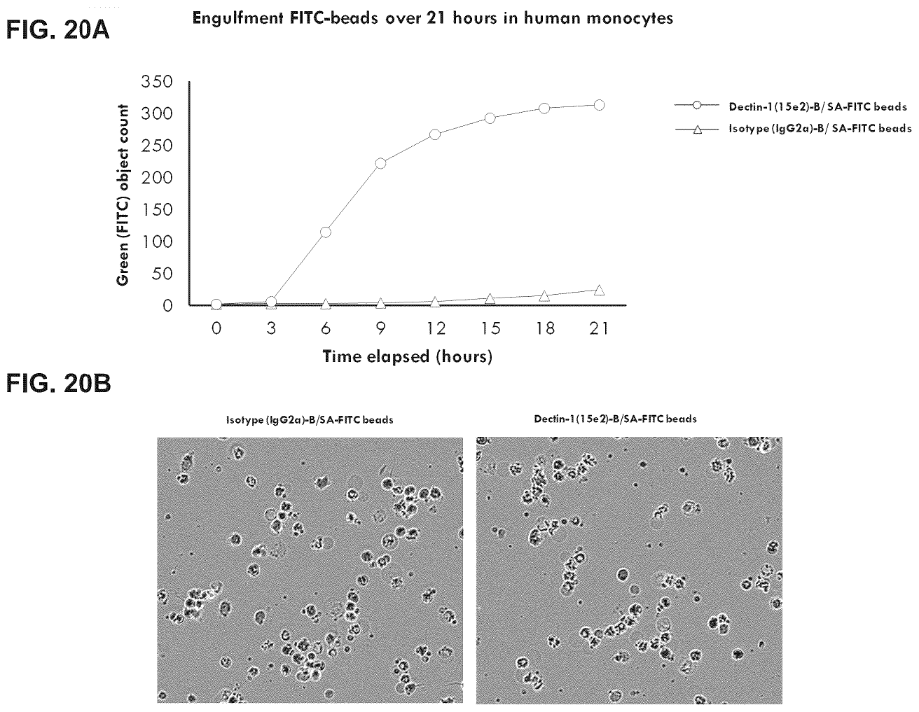

Engulfment of 40 nm beads by primary human monocytes was also examined. FIGS. 20A & 20B show engulfment of streptavidin FITC-labeled polystyrene beads (40 nm) conjugated with biotinylated Dectin-1 antibody (15e2-B) or biotinylated isotype (IgG2a-B) by human monocytes. Polystyrene FITC beads were saturated with biotinylated Dectin-1 antibody or isotype control for 30 minutes. The antibody/bead complexes were then incubated with cultured human monocytes at a ratio of 1:6 (cells:beads). FITC staining of monocytes was monitored by IncuCyte live cell imaging. FIG. 20A shows engulfment of SA-FITC beads by monocytes over 21 hours, quantified by the IncuCyte analysis software and expressed as green (FITC positive) object count. FIG. 20B shows representative images of FITC positive cells at 15 hours of the experiments.

Anti-Dectin-1 antibody was found to promote the engulfment of very small polystyrene beads (40 nm). These data show that very small particles can be engulfed by targeting Dectin-1 and indicate the possibility to promote phagocytosis of very small disease-causing agents such as viruses.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

### REFERENCES

Henson, P. M., and Bratton, D. L. “Recognition and removal of apoptotic cells. In Phagocyte-pathogen interactions: macrophages and the host response to infection, D. G. Russell and S. Gordon, eds. (ASM Press) 2009, pp. 341-365.
Flannagan, R. S., Jaumouille', V., and Grinstein, S. “The cell biology of phagocytosis”, 2012, Annu. Rev. Pathol. 7, 61-98
Gordon S, “Phagocytosis: An Immunobiologic Process” 2016 Immunity 44, 463-475
Taylor P R, et al. “The β-glucan receptor, dectin-1, is predominantly expressed on the surface of cells of the monocyte/macrophage and neutrophil lineages”. J. Immunol. 2002; 169:3876-3882.
Herre J, Marshall A S J, Caron E, Edwards A D, Williams D L, Schweighoffer E, Tybulewicz V, Reis e Sousa C, Gordon S, and Brown G D, “Dectin-1 uses novel mechanisms for yeast phagocytosis in macrophages” Blood, 2004, Vol 104, No 13, 4038-45
Rosales C and Uribe-Querol E, “Phagocytosis: A Fundamental Process in Immunity” 2017, BioMed Research International, Volume 2017, Article ID 9042851, 18 pages
Ackerman M E, Moldt B, Wyatt R T, Dugast A S, McAndrew E, Tsoukas S, Jost S, Berger C T, Sciaranghella G, Liu Q, Irvine D J, Burton D R, Alter G, “A robust, high-throughput assay to determine the phagocytic activity of clinical antibody samples”, J. Immunol. Methods, 2011, 366, pp. 8-19.

1. A method of removal or reducing the number of a disease-causing agent by targeted phagocytosis in a human subject comprising administering to said subject a binding protein comprising a first binding domain that specifically binds to the agent, a second binding domain that binds to a phagocytotic receptor, Dectin-1, expressed on a myeloid cell and induces phagocytosis activity of the myeloid cell, and an immunoglobulin Fc domain.

2. The method of claim 1, wherein the administration of the antibody reduces the number of the disease-causing agent below the limit of detection and the level remains below detection for at least about 1 week after dosing of the antibody.

3. The method of claim 1, wherein the reduction of the disease-causing agent takes place within the first 24 hours or 48 hours after administration.

4. The method of claim 1, wherein the reduction of the disease-causing agent is reversible.

5. The method of claim 1, wherein said reduction of the disease-causing agent leads to a reduction in symptoms.

6. The method of claim 1, wherein the method is used to remove disease-associated protein and protein aggregates to inhibit aberrant protein accumulation and therefore alleviating or preventing progression of the disease including neurodegenerative, fibrosis or amyloidoses.

7. The method of claim 1, wherein the method is used to remove or reduce level of cancer, tumor or lymphoma cells and therefore inhibit or prevent progression of the disease.

8. The method of claim 1, wherein the method is used to remove or reduce level a microbe (e.g., bacteria, fungus, virus), a protozoan parasite and therefore inhibit or prevent progression of the disease.

9. The method of claim 1, wherein the method is used to remove or reduce level of senescent cells and their products and therefore inhibit or prevent progression of ageing.

10. (canceled)

11. The method of claim 1, wherein the method is used to remove or reduce level of LDL and other agents that induce cardiovascular diseases including arteriosclerosis or familial hypercholesterolemia and therefore inhibit or prevent progression of the diseases.

12. The method of claim 1, wherein the method is used to remove or reduce level of mast cells and therefore inhibit or prevent progression of allergy, fibrosis, COPD, asthma and other mast cells related disease including immunoproliferative diseases.

13. The method of claim 1, wherein the method is used to remove or reduce level of eosinophils and therefore inhibit or prevent progression of allergy, fibrosis, COPD, asthma and other eosinophil related disease including immunoproliferative diseases.

14. The method of claim 1, wherein the method is used to remove or reduce level of ILC2 cells and therefore inhibit or prevent progression of allergy, fibrosis, COPD, asthma and other ILC2 cells related disease including immunoproliferative diseases.

15. The method of claim 1, wherein the method is used to remove or reduce level of inflammatory immune cells in muscles, GI tract, lungs, heart, joints, brain and other organs and therefore inhibit or prevent progression of myositis, IBD, RA, allergy, fibrosis, COPD, asthma and other immune cells related disease including immunoproliferative diseases.

16. The method of claim 1, wherein the binding protein is selected from specific antibodies; two IgGs covalently linked; IgG-scFv; intrabodies, peptibodies, nanobodies, single domain antibodies, SMTPs, and multispecific antibodies (e.g., bispecific antibodies, diabodies, triabodies, tetrabodies, tandem di-scFV, tandem tri-scFv, ADAPTIR); Fab, Fab′, F(ab′)2, and Fv fragments, Fab′-SH, F(ab′)2, diabodies, linear antibodies, scFv antibodies, VH, and multispecific antibodies formed from antibody fragments.

17. The method of claim 16, wherein the binding domains of the binding protein is non-human, chimeric, humanized, or human, preferably humanized or human.

18. The method of claim 1, wherein the binding protein is a bispecific antibody comprising a first binding domain that binds to Dectin-1 and a second binding domain that binds to a target antigen expressed by the disease-causing agent.

19. The method of claim 1, wherein the myeloid cell is a monocyte, macrophage, or dendritic cell.
